SEQUENCE LISTING (3 sequences)

SEQ ID NO: 1 - protein, 7 aa, Artificial sequence
  fragment derived from tenascin C
PLAEIDG

SEQ ID NO: 2 - protein, 15 aa, Artificial sequence
  fragment derived from tenascin C
GGGPLAEIDGIELTY

SEQ ID NO: 3 - protein, 18 aa, Artificial sequence
  fragment derived from tenascin C
RLIEDICLPRWGCLWEDD

(19) United States
(12) Patent Application Publication
(10) Pub. No.: US 2016/0029948 A1
(43) Pub. Date: Feb. 4, 2016

(54) FUNCTIONAL NEAR-INFRARED FLUORESCENCE LYMPHATIC MAPPING FOR DIAGNOSING, ACCESSING, MONITORING AND DIRECTING THERAPY OF LYMPHATIC DISORDERS

(71) Applicant: BOARD OF REGENTS OF THE UNIVERSITY OF TEXAS SYSTEM, Austin, TX (US)

(72) Inventors: Eva M. SEVICK-MURACA, Montgomery, TX (US); Caroline E. FIFE, The Woodlands, TX (US); Milton V. MARSHALL, Houston, TX (US); Erik A. MAUS, Houston, TX (US); John C. RASMUSSEN, Spring, TX (US); I-Chih TAN, Houston, TX (US)

(21) Appl. No.: 14/818,571

(22) Filed: Aug. 5, 2015

Related U.S. Application Data
(63) Continuation of application No. 13/897,924, filed on May 20, 2013, now abandoned.
Application No. 13/897,924 is a continuation of application No. 12/886,770, filed on Sep. 21, 2010, now abandoned.
(60) Provisional application No. 61/244,302, filed on Sep. 21, 2009.

(51) Int. Cl. A61B 5/00

(52) U.S. Cl. CPC A61B 5/414; A61B 5/0071; A61B 5/418

(57) ABSTRACT

Methods and imaging agents are used to functionally image lymph structures and to identify, diagnose, assess, monitor and direct therapies for lymphatic disorders. Embodiments of the methods utilize highly sensitive optical imaging and fluorescent spectroscopy techniques capable of rapid temporal resolution to non-invasively track or monitor packets of imaging agents flowing in one or more lymphatic structures in human patients to provide quantitative information regarding lymph propulsion and functionality of the lymphatic structures. An imaging agent comprises a fluorophore labeled peptide capable of binding integrin α₉β₁ on a lymphatic structure.

### CROSS-REFERENCE TO RELATED APPLICATION

This application is a continuation of U.S. patent application Ser. No. 13/897,924 filed May 20, 2013, which was a continuation of Ser. No. 12/886,770 filed Sep. 21, 2010 claims the benefit under 35 U.S.C. §119(e) of U.S. Provisional Patent Application No. 61/244,302 filed Sep. 21, 2009, the disclosure of which is hereby incorporated herein by reference.

### STATEMENT REGARDING FEDERALLY SPONSORED RESEARCH OR DEVELOPMENT

This invention was made with U.S. Government support under Grant Nos. CA112679, HL092923, CA128919, CA136404 awarded by the National Institutes of Health. The government has certain rights in the invention.

### TECHNICAL FIELD

This disclosure relates to the use of biomedical imaging, and more particularly to methods and compositions for functionally imaging and measuring lymphatic architecture and lymph propulsion and transport in an individual. More particularly, this disclosure relates to the use of such methods and compositions in determining differences between normal and aberrant lymphatic structure and function, using near-infrared (NIR) fluorescence techniques, for diagnosing, assessing and monitoring disorders of the lymphatic system and in directing, facilitating and evaluating treatments and therapies for lymphatic disorders.

### BACKGROUND

The lymphatic system, comprising lymphatic vessels, nodes, and lymph fluid and materials carried by it, has several functions. It is involved in maintenance of bodily fluid and protein, immunity and digestion. A properly functioning lymphatic system collects the fluid and protein that exits the circulatory system through capillaries and returns it to the circulatory system. Without this activity, the loss of fluid would rapidly become life threatening. The lymphatic system also plays an integral role in the function of the immune system. It is the first line of defense against disease. The network of vessels and nodes transports and filters lymph fluid containing antibodies and lymphocytes as well as infectious agents such as microorganisms. Lymph vessels in the lining of the gastrointestinal tract absorb fats from food and a malfunction of this part of the lymphatic system can result in malnutrition. The lymphatic system therefore also impacts diseases such as excessive obesity caused by abnormal fat and carbohydrate metabolism.

The lymphatic system comprises vessels or ducts that begin in tissues and are designed to carry lymph fluid to local lymph nodes where the fluid is filtered and processed and sent to the next lymph node down the line until the fluid reaches the thoracic duct where it enters the blood stream. Lymph vessels infiltrate all tissues and organs of the body. Lymph fluid is generated from capillaries which, because of tissue motion and hydrostatic pressure, enters the lymph vessels carrying with it local and foreign substances and materials from the tissues

Lymphedema may be inherited (primary) or caused by injury to the lymphatic vessels (secondary). Congenital or primary lymphedema afflicts 1 in every 6,000 newborns and can also appear at the onset of puberty. Acquired or secondary lymphedema is caused by the filaria parasite (in a condition referred to as elephantiasis) or by trauma due to radiation therapy, infiltrating cancer, surgery, or infection. In developing-world countries, 100 million people are afflicted worldwide by filariasis. However, in Western countries, acquired lymphedema afflicts 3 to 5 million people. The etiology for trauma-associated, acquired lymphedema is thought to arise from the interruption of lymph channels coupled with postsurgical infection or radiation-induced skin reaction. The symptoms may occur at any time following the initial trauma, striking at a rate cited between 6 and 62.5% of breast cancer survivors who have undergone axillary lymph node dissection, up to 64% of all patients who undergo groin dissections, and 25% of all radical hysterectomy patients. Little is known about the molecular or functional basis of acquired lymphedema or which persons could be at risk for the condition. There is a paucity of strategies for predicting or managing lymphedema due in part to the lack of diagnostic imaging approaches to noninvasively and routinely measure lymphatic function. Since lymph function is also implicated in diseases of significant prevalence (e.g., diabetes, obesity, cancer, and asthma), the ability to quantitatively image lymph function could have substantial impact on the health of the world's population.

Treatment for lymphedema is generally limited to compression bandaging and manual lymph drainage or massage to limit setting and encourage lymph drainage. This accepted method to manage lymphedema is through the use of a non-surgical and non-pharmacological technique called complete decongestive therapy (CDT), which includes manual lymph drainage (MLD), compression bandaging, therapeutic exercise, and meticulous skin care. Although its effectiveness remains controversial, MLD consists of a massage-like technique that is performed for 30-60 minutes to first stimulate lymphatic drainage from receiving lymph node basins, and then presumably stimulate contractile or “pumping” function of the superficial (epifascial) lymphatic system for subsequent drainage. Response to MLD is usually measured indirectly through reduction of limb volume using a number of accepted and experimental methods over a period of weeks to months. Hence there is no method available to immediately evaluate efficacy of MLD. While CDT response rates using limb volumetric measurements are reported to be 67.7% for lower extremity and 59.1% for upper extremity lymphedema subjects over a management period of 12 months (Ko D S, Lerner R, Klose G, Cosimi A B. Effective treatment of lymphedema of the extremities. Arch Surg. April 1998; 133(4):452-458), there remains no method (i) to predict who will respond to CDT or (ii) to measure whether contractile or “pumping” function is indeed enhanced by MLD. Lymphedma is often a lifelong problem requiring daily treatment. In more extreme situations surgical techniques for correcting lymphedema may involve procedures involving excision such as: circumferential excision of the lymphedematous tissue followed by skin grafting (Charles technique); longitudinal removal of the affected segment of skin and subcutaneous tissue and primary closure (Homans technique); excision of subcutaneous tissue and tunneling of a dermal flap through the fascia into a muscular compartment of the leg (Thompson technique) or physiological procedures such as lympholymphatic anastomosis (autologous lymphatic grafts to bridge obstructed lymphatic segments); lymphovenous shunt (anastomosis of lymphatic channels to veins); lymphangioplasty enteromesenteric flap omental transfer (pedicled portion of omentum transposed to the affected limb).

Unfortunately, the phenotype of lymphatic architecture and function in both humans and models of disease has not been well characterized due to the lack of in vivo imaging techniques with sufficient temporal and spatial resolution. Aberrant lymph architecture is difficult to routinely assess (for review see Sharma, R., J. A. Wendt, J. C. Rasmussen, et al., New horizons for imaging lymphatic function. Ann N Y Acad Sci, 2008. 1131: p. 13-36) because lymph provides little endogenous contrast and thus cannot be effectively probed directly using ultrasound, MR or CT techniques. Thus, as with MR or CT angiography, milliliters of contrast agent are required and lymphatic vasculature is not readily accessible and requires a potentially painful and damaging cannulation of lymphatic vessels. In addition, MR and CT require large and expensive imaging equipment and instrumentation.

Currently, the clinical gold standard for lymphatic imaging is lymphoscintigraphy which consists of an intradermal or subcutaneous injection of a radiocolloid contrast agent, typically 99m-Tc, followed by imaging with a gamma camera. The procedure can be painful, is time-consuming requiring several minutes to acquire images, exposes the patient to ionizing radiation, and exhibits poor resolution. Thus, while gross lymph architecture such as main vessels and nodes are visualized in the scintigrams, the long integration times associated with gamma cameras prevent imaging of lymphatic function and the image resolution limits visualization of fine lymphatic vasculature.

As described in, among others, U.S. Pat. Nos. 5,865,754; 7,054,002; 7,328,059; US Patent Application Publication Nos: 2007/0286468; 2008/0056999; 2008/0064954; 2008/0175790; and Sevick-Muraca, E. M., R. Sharma, J. C. Rasmussen, et al., Imaging of lymph flow in breast cancer patients after microdose administration of a near-infrared fluorophore: feasibility study. Radiology, 2008. 246(3): p. 734-41 as well as recently reviewed, in Rasmussen et al., “Lymphatic imaging in humans with near-infrared fluorescence,” Curr Opin Biotechnol. February 2009; 20(1):74-82, the use is described of non-invasive imaging of active lymph drainage following intradermal administration of microgram amounts of indocyanine green (ICG), a green dye used for hepatic clearance and ophthalmological indications, by using its near-infrared (NIR) fluorescence properties for optical imaging.

There are presently very few technologies with the ability to non-invasively image the lymphatic system in vivo and in real-time, and there is a paucity of imaging technologies with the sensitivity and temporal resolution to discriminate lymphatic function. Consequently, there is continuing interest in non-invasive imaging methods and imaging agents for dynamically assessing lymph function in vivo to facilitate, direct and evaluate therapies for the treatment of lymphatic disorders.

### SUMMARY

Methods and imaging agents for functional imaging of lymph structure and in human patients are disclosed herein. In some embodiments, highly sensitive optical imaging and fluorescent spectroscopy techniques are used to track or monitor packets of imaging agent (e.g., organic, soluble dyes) being propelled through one or more lymphatic structures. The packets of organic dye may be tracked to provide quantitative information regarding lymph propulsion and function. Thus, the disclosed methods provide non-invasive ways of assessing lymph function in deep lymph structures. The organic dyes may be excited at the near-infrared wavelength region of 750-800 nm with fluorescence >800 nm allowing for deep tissue imaging of lymphatic function. Unlike some prior lymphatic imaging techniques, embodiments of the present methods provide greater sensitivity and temporal resolution permitting discrimination of lymphatic function, and the ability to interrogate the difference between normal and aberrant lymphatic structure and function.

In some embodiments, a control comprises a measurement obtained from one or more normal individuals (e.g., known to be apathogenic or unaffected by a lymphatic disease or other aberrancy).

In accordance with certain embodiments, a method is provided to non-invasively assess lymphatic structure and function in an individual. This method comprises administering at least one imaging packet to a lymph structure of the individual, the imaging packet containing at least one contrast agent (e.g., one or more dyes) having a characteristic excitation wavelength and a characteristic fluorescence emission wavelength; noninvasively illuminating a tissue surface of a region of interest on the individual's body with radiation at the characteristic excitation wavelength such that the dye or dyes in an imaging packet fluoresce. The fluorescence emissions are noninvasively detected and, over time, fluorescence images are obtained. These images are used to obtain parameters of lymph propulsion within the lymphatic architecture and to visualize lymphatic structures and pathways.

In certain embodiments, a plurality of images are obtained over time (e.g., in “real time”) in a predetermined location and distance and by using the fluorescence images to track the location of each such packet in the region of interest as a function of time. By doing so, one can determine a lymph propulsion and functional measurement.

The lymph propulsion and functional measurement are defined as: (i) the frequency of propulsion of “packets,” (ii) the velocity of the “packets,” (iii) the number of lymphatic structures (or lymph vessel density), (iv) the architecture of vessels that “packets” travel in (dilated vessels, tortuous vessels), (v) the permeability or “leakiness” of the lymphatic vessels, and other parameters that may describe the functional status of the lymphatics. The same imaging of process of following “packets” of dye can then be repeated on unaffected regions, for example the opposite limb of the same individual or the same region of tissue on a normal control individual, By comparing the initial lymph propulsion measurement and lymphatic architecture with that obtained using unaffected tissues from the same individual or the same region of tissue on a normal control individual, one can identify the presence of a lymphatic disorder and thus this method has use in diagnosing lymphatic disorders. Comparing the initial lymph propulsion or architecture measurement to a subsequently determined lymph propulsion measurement for the region of interest with that obtained post-therapy or treatment facilities allows one to determine the effect of therapy and monitor the patients progress on a treatment regimen. Thus in some embodiments, the methods can be employed to guide treatment designed to ameliorate a lymphatic dysfunction. For example, by understanding where functional lymphatic (i.e., those lymphatic structures that transport “packets” of dye) are located in tissues, one could direct therapies and treatments. Such treatments include but are not limited to manual lymph drainage (MLD) for treatment of, among other disorders, lymphedema, wherein the drainage of fluid can be directed toward functional lymphatics identified by the “packets.” Other treatments may include anti-cancer metastasis therapies upon visualizing lymphatic changes that are present in the beginning stages of cancer metastasis.

The same process can then be repeated on unaffected regions, for example the opposite limb of the same individual, or a similar region of tissue on a normal, control individual. By comparing an initial lymph propulsion measurement with that obtained using unaffected tissues from the same individual, or a similar region of tissue on a normal (control) individual, one can identify the presence of a lymphatic disorder. Thus, in some embodiments this method has use in diagnosing lymphatic disorders. In some embodiments, a control comprises measurements obtained from one or more normal individuals (e.g., known to be apathogenic or unaffected by a lymphatic disease, dysfunction or aberrancy). In certain embodiments, comparing an initial lymph propulsion measurement to a subsequently determined lymph propulsion measurement for the region of interest with that obtained post-therapy or post-treatment allows the healthcare practitioner to determine the effect of the therapy or other treatment. In certain embodiments, an above-described method provides a way to monitor the patient's progress on a treatment regimen. Thus, in some embodiments an above-described method is potentially employed to guide treatment designed to ameliorate a lymphatic dysfunction. Such treatments include but are not limited to manual lymph drainage (MLD) for treatment of, among other disorders, lymphedema.

In certain embodiments, an above-described method of non-invasively assessing lymph function in an individual comprises administration of at least one imaging packet to a lymph structure of the individual, the imaging packet containing at least one contrast agent having a characteristic excitation wavelength and a characteristic fluorescence emission wavelength. In some embodiments of an above-described method, measuring lymph propulsion comprises at least one of lymph pulse frequency (i.e. the inverse time between the appearance of a lymph “packet” at a single location in the lymphatic structure) and lymph flow velocity of a single “packet.” In some embodiments, the lymphatic disorder can be lymphangiogenesis (such as that caused by cancer metastasis, injury, infection or genetic disorder, for example) or lymphedema.

In some embodiments, an above-described method comprises tracking the location of each packet and capturing each image at an integration time ranging from about 10 milliseconds to about 1 second. In certain embodiments, the integration time is about 200 milliseconds. In some embodiments, the characteristic excitation wavelength is in the region of 750-800 nm, and the characteristic fluorescence emission wavelength is greater than 800 nm.

In accordance with certain embodiments, a method of non-invasively assessing lymph function in an individual is disclosed which comprises performing functional NIR fluorescence imaging of at least one lymphatic structure in the individual. In some embodiments the method includes a) administering at least one imaging packet to a lymph structure of the individual, the imaging packet containing at least one imaging agent having a characteristic excitation wavelength and a characteristic fluorescence emission wavelength; b) noninvasively illuminating a tissue surface of a first region of interest on the individual's body with radiation at the characteristic excitation wavelength; c) noninvasively detecting fluorescence emissions from each such imaging packet and capturing a plurality of fluorescence images for an interval of time; d) using the fluorescence images to visualize lymph structures in the first region of interest and to track the location of each such packet in the first region of interest as a function of time to obtain a set of tracked image locations as a function of time; e) determining from the tracked locations as a function of time an initial lymph propulsion measurement; f) comparing the initial lymph propulsion measurement to a subsequently determined lymph propulsion measurement or to a control; and g) determining from the results of f) the functionality of a lymph structure in the first region of interest in the individual.

In some embodiments of an above-described method, in e) the lymph propulsion measurement comprises at least one of lymph pulse frequency and lymph flow velocity. In some embodiments, in e), the initial lymph propulsion measurement comprises an initial lymph flow velocity; f) comprises comparing the initial velocity to a subsequently determined lymph flow velocity; and in g), determining the functionality of a lymph structure includes determining that lymphatic function in the region of interest is improved if the subsequent lymph flow velocity is greater than the initial lymph flow velocity.

In some embodiments, of an above-described method in e), the initial lymph propulsion measurement comprises an initial lymph pulse frequency; f) comprises comparing the initial lymph pulse frequency to a subsequently determined lymph pulse frequency; and in g), determining the functionality of a lymph structure includes determining that lymphatic function in the region of interest is improved if the subsequent lymph pulse frequency is greater than the initial lymph pulse frequency. In some embodiments, in g) a lymph propulsion measurement of less than a control value is indicative of lymphedema.

In some embodiments, an above-described method includes, prior to performing f), administering to the individual a treatment (e.g., manual lymph drainage) to ameliorate a lymphatic dysfunction. In some embodiments, in f), such control comprises at least one lymph propulsion measurement of a corresponding region of interest of an individual or group of individuals known to be apathogenic or unaffected by a lymphatic disease, dysfunction or aberrancy. In some embodiments, in f), such control comprises at least one lymph propulsion measurement of a second region of interest in the individual, wherein the second region of interest contains apparently normally functioning lymphatic structures.

In some embodiments, a disclosed method includes h) identifying a lymphatic disorder in the individual based on the results of the determination in (g). In some embodiments, in f), the comparison of the initial lymph propulsion measurement to a subsequently determined lymph propulsion measurement of the first region of interest indicates a change in lymph function over time.

In some embodiments, the imaging agent comprises a peptide capable of selectively binding to integrin α₉β₁ on a lymph vessel endothelium, and a near-infrared fluorophore conjugated to the peptide and having characteristic excitation wavelength and a characteristic fluorescence emission wavelength. In some embodiments, the imaging agent comprises a near-infrared fluorophore having a characteristic excitation wavelength and a characteristic fluorescence emission wavelength, the fluorophore conjugated to a peptide capable of selectively binding to a protein that is taken up and retained in the lymphatics to a greater extent than an unconjugated fluorophore.

Also provided in accordance with certain embodiments are new imaging agents targeted to lymph endothelial cell integrin α₉β₁. In certain embodiments, the imaging agents comprise a peptide derived from the extracellular matrix protein tenascin C sequence, which is known to bind to the lymph endothelial cell integrin α₉β₁. The peptide is labeled or conjugated with a near-infrared fluorophore and used to detect lymphangiogenesis in vivo as well as activation of lymphatic endothelial cells in vitro. Lymph endothelial cell integrin α₉β₁ expression may be related to the beginnings of tumor metastasis and/or lymphangiogenesis. As such, embodiments of the imaging agents may be used to stain lymph structures for detailed imaging of lymph architecture as well as serving as potential markers for tumor lymphangiogenesis, tumor metastases, infection, progressive disease, and the like.

In some embodiments an above-described method or composition is used to determine activation of lymphatic endothelial cells, such as that which is indicative of lymphangiogenesis. In some embodiments an above-described method or composition is used to identify or diagnose a lymphatic disorder in human or non-human mammal. In some embodiments an above-described method or composition is used to identify a lymphatic disorder in a limb of a patient. In some embodiments an above-described method or composition is used to identify a patient, or limb of a patient at risk of developing a lymphatic disorder. In some embodiments an above-described method or composition is used to monitor a lymphatic disorder in a patient.

In some embodiments an above-described method or composition is used to direct therapy or treatment for a lymphatic disorder in a patient. In some of these embodiments, the therapy includes an anti-metastatic or anti-lymphangiogenic agent directed at VEGF, NPR2, EpCAM, alpha 9 integrins, or an inhibitor or activator of signaling associated with lymphangiogenesis. Thus, in certain embodiments the patient's lymphatic disorder is metastatic cancer or a vascular disease. In certain other embodiments, the lymphatic disorder is lymphangiogenesis or lymphedema. The above-described methods and compositions are potentially applicable to all mammals but have particular utility in humans.

In accordance with some embodiments, a method to aid in diagnosing a lymphatic disorder is provided which comprises performing an above described method and h) determining increased likelihood of a lymphatic disorder in the individual if the determination in g) indicates reduced functionality of a lymph structure in the individual compared to the control or to the subsequently determined lymph propulsion measurement. Some embodiments also include i) administering a treatment for a lymphatic disorder based on the results of (h).

In accordance with some embodiments, a method to aid in directing treatment of an individual for a lymphatic disorder is provided which comprising performing an above described method, and h) identifying at least one aberrant lymphatic structure in the individual in need of treatment of the lymphatic disorder if the determination in g) indicates reduced functionality of a lymph structure in the individual compared to the control or to the subsequently determined lymph propulsion measurement. In certain embodiments, the method comprises i) determining whether to administer a therapeutic agent to the individual based on the results of the identification in (h).

In accordance with some embodiments, a method of detecting activation of lymphatic endothelial cells in vivo comprises a) administering to a lymph structure of an individual in need of such detecting, at least one imaging packet comprising a near-infrared fluorophore having a characteristic excitation wavelength, the fluorophore conjugated to a peptide capable of selectively binding to integrin α₉β₁ on a lymph vessel endothelium, and a pharmacologically acceptable carrier; b) noninvasively illuminating a tissue surface of a region of interest on the individual's body with radiation at the characteristic excitation wavelength; and c) noninvasively detecting fluorescence emissions from the fluorophore-peptide conjugates selectively bound to a lymph vessel endothelium in the region of interest, as an indication of lymphatic endothelial cell activation. In some embodiments, in c), such indication of lymphatic endothelial cell activation indicates lymphangiogenesis in the region of interest. These and other embodiments, features and advantages will be apparent in the detailed description and drawings which follow.

### BRIEF DESCRIPTION OF THE DRAWINGS

FIG. 1: Images of lymphatics of control subjects in (a) left hand after interdigital injections (subject C, 16), (b) right elbow (subject C20), (c) upper right arm (subject C24), and (d) entire right arm from wrist to shoulder (subject C24). Black square regions are covered injection sites to prevent oversaturation of the detection system;

FIG. 2: Images of lymphatics of control subjects in (a) left foot, (b) lower leg (subject C15), (c) back of knee (subject C23), and (d) thigh (subject C05);

FIG. 3: NIR fluorescence images of the (a) cubital (subject C, 16), (b) axillary (subject L20), (c) popliteal (subject C21), and (d) inguinal (subject C09) lymph nodes. The additional fluorescent pathways in (d) are draining two additional injections, made just above and below the right buttock, into the inguinal nodes;

FIG. 4: Comparison of symptomatic (a, c, and e) and asymptomatic (b, d, and f) arms of three subjects. (a) Symptomatic hand with extravascular fluorescence from injection sites and some networks of fluorescent lymphatic capillaries and (b) normal looking asymptomatic hand (subject L11). (c) Symptomatic arm with extravascular fluorescence and tortuous vessels and (d) normal looking asymptomatic arm (subject L18). (e) Symptomatic arm with extravascular fluorescence and (f) asymptomatic arm with fluorescent lymphatic capillaries, tortuous lymph vessels and lymph reflux (subject L05) (See FIG. 6). (g) Back and (h) front of symptomatic hand in which active lymphatic propulsion was seen pushing fluid into hand (subject L02). Bright spot on palm (h) is a spontaneous fistula or weep hole that developed to permit lymph to drain;

FIG. 5: (a) A closer view of the lymph vessel with lymph reflux in the asymptomatic arm of subject L05 seen in FIG. 5f. In this example there appears to be insufficient valvular function allowing a portion of each forward pulse to drain back from region of interest (ROI). ROI 2 (red) to ROI 1 (blue) as illustrated in (b) a three dimensional plot of fluorescent intensity as a function of time and distance. By following the intensity peaks the direction of flow is determined. (c) shows the fluorescent intensity profiles at ROI 1 and ROI 2;

FIG. 6: Comparison of symptomatic and asymptomatic legs of subject L10 (a, b, and c) and subject L17 (d). (a) Lower legs of a 23 year old female (subject L10) with lymphedema on the left leg (on right in images). Asymptomatic limb looks normal though it has a paucity of lymphatic vessels while the symptomatic limb has diffuse extravascular fluorescence with no distinct structure. (b) A closer look at the ankles of the same subject as (a) shows that the asymptomatic limb has some tortuous channels emanating from the injections site while the symptomatic limb shows diffuse extravascular fluorescence again with perhaps a large curved lymphatic vessel just above the ankle bone. (c) Image illustrating the diffusion of dye into the symptomatic foot of the same subject. (d) Lower legs of a 65 year old female (subject L17) with clinically diagnosed lymphedema on the right leg (on left in image). Both symptomatic and asymptomatic legs show extensive diffuse extravascular fluorescence, though some structure is apparent in asymptomatic limb;

FIG. 7: Images of (a) extravascular fluorescence in the symptomatic forearm of subject L18 and (b) tortuous lymphatic capillaries in the asymptomatic thigh of subject L13;

FIG. 8: NIR fluorescent images of (a) right arm of control subject N06, (b) symptomatic arm with lymphatic vessels and extravascular fluid in LE subject L02, (c) lymphatic dermal backflow in the same symptomatic hand in 1(b), (d) right elbow in control subject N06 [the same arm in 1(a)], (e) symptomatic elbow with hyperplastic networks and extravascular lymphatic fluid in LE subject L02 [the same arm in 1(b)], and (f) asymptomatic arm with hyperplastic networks in the same LE subject L02;

FIG. 9: NIR fluorescent images of (a) right foot in control subject N11, (b) symptomatic leg with lymphatic vessels and extravascular fluid in LE subject L07, (c) asymptomatic leg in the same LE subject L07, (d) symptomatic foot in LE subject L10 (e) bottom of both feet with dermal backflow on symptomatic foot in the same LE subject L10, and (f) the same symptomatic foot with extravascular fluid and lymphatic backflow;

FIG. 10: Bar graphs showing average apparent lymph velocities and propulsion periods on different arms in Lymphedema (symptomatic and asymptomatic) and control (left and right) subjects pre- and post-MLD (*: p<0.05);

FIG. 11: Bar graphs showing average apparent lymph velocities and propulsion periods in arms from different groups (symptomatic, asymptomatic, and control) pre- and post-MLD. The statistically significant changes in percentage from pre- to post-MLD are labeled (*: p<0.05);

FIG. 12: Bar graphs showing average apparent lymph velocities and propulsion periods on different legs in Lymphedema (symptomatic and asymptomatic) and control (left and right) subjects pre- and post-MLD (*: p<0.05);

FIG. 13: Bar graphs showing average apparent lymph velocities and propulsion periods in different group (symptomatic, asymptomatic, and control) of legs pre- and post-MLD. The changes in percentage from pre- to post-MLD are labeled (*: p<0.05).

FIG. 14: Injection sites on the face and neck to probe function lymph drainage in case study subject in which knowledge of where lymphatic drainage pathways are desired to direct MLD.

FIG. 15: Front view of case study subject after injection of ICG and during imaging and reference photograph.

FIG. 16: Sites of injection in the arms of a patient with history of congestive heart failure (CHF), Hodgkin's disease, radiation treatment, and early lymphedema symptoms.

FIG. 17: Abnormal lymphatics in the right upper edematous arm of a patient with history of congestive heart failure (CHF), Hodgkin's disease, and radiation treatment after injection of ICG in sites depicted in FIG. 16. The tortuous vessels transport “dye” but not in a propulsive action. Instead, they drain into the main lymphatic vessels or “trunk” that actively propels “packets” of dye in a fashion normally seen in healthy patients to the axilla.

FIG. 18: Images showing retrograde flow in asymptomatic arm of the same subject as in FIG. 17 suffering from early lymphedema.

FIG. 19: Fluorescence microscopy of LECS stimulated by VEGF-C to express α₉β₁ on the extracellular membrane as stained by (from left to right, top) by (i) DAPI, a nuclear stain, (ii) IRDye800-GGGPLAEIDGIELTY (SEQ ID NO: 2), the imaging conjugate that targets α₉β₁, and (iii) a fluorescently labeled antibody that targets the 131 portion of α₉β₁. The bottom figures shows the overlay of all the fluorescence micrographs indicating colocalization of α₉β₁ and IRDye800-GGGPLAEIDGIELTY (SEQ ID NO: 2).

FIG. 20: NIR fluorescent image of an animal model of lymphangiogenesis (top) with a MATRIGEL plug impregnated with LEC stimulating factor HGF on the right with a control MATRIGEL plug without HGF on the left. The animal image (top) is taken 24 hours after administration of IRDye800-GGGPLAEIDGIELTY (SEQ ID NO: 2) showing agent uptake into the MATRIGEL plug containing LEC stimulating factor HGF and little or no uptake into the MATRIGEL plug without HGF. When the MATRIGEL plugs were removed from the animal and imaged, they were then imaged for the presence of IRDye800-GGGPLAEIDGIELTY (SEQ ID NO: 2). The plug containing HGF showed fluorescence due to IRDye800-GGGPLAEIDGIELTY (SEQ ID NO: 2) due presumable to LEC activation associated with lymphangiogenesis while the control plug shows none or little.

FIG. 21: NIR imaging shows the lymphatic architecture in a mouse treated to induce skin inflammation on the right side by topical application of Oxazolone. The NIR imaging was conducted with intradermal administration of ICG (as shown above in human studies) to image both the right and left side lymphatics. The dorsal view (top) shows a greater number of lymphatic vessels on the right side that is associated with inflammation and lymphangiogenesis. Increased vessel density, leakiness, and dilated vessels on the inflamed side as compared to the untreated side of the animal as evident from the lymphatic imaging as compared to the left, untreated side.

FIG. 22: NIR imaging of the same mouse shows that the lymphatic architecture in a mouse treated to induce skin inflammation on the right side by topical application of Oxazolone is also imaged by α₉β₁ integrin targeting, IRDye800-GGGPLAEIDGIELTY (SEQ ID NO: 2). The right side of the animal which has greater lymphatic density as indicated by ICG imaging (as shown in FIG. 21) is also associated with increased IRDye800-GGGPLAEIDGIELTY (SEQ ID NO: 2) fluorescence presumably owing to the stimulation of LECS in the process of lymphangiogenesis to produce α₉β₁ integrin.

### DETAILED DESCRIPTION

The following discussion is directed to various embodiments of the invention. Although one or more of these embodiments may be preferred for some applications, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

### DEFINITIONS

In this disclosure, the use of the singular includes the plural, the word “a” or “an” means “at least one,” and the use of “or” means “and/or,” unless specifically stated otherwise. Furthermore, the use of the term “including,” as well as other forms, such as “includes” and “included,” is not limiting. Also, terms such as “element” or “component” encompass both elements or components comprising one unit and elements or components that comprise more than one unit unless specifically stated otherwise.

The term “about” when referring to a numerical value or range is intended to include larger or smaller values resulting from experimental error that can occur when taking measurements. Such measurement deviations are usually within plus or minus 10 percent of the stated numerical value.

Temperatures, ratios, concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range, as if each numerical value and sub-range is explicitly recited. For example, a camera integration time range of about 750 nm to about 10 milliseconds to about 1 second should be interpreted to include not only the explicitly recited limits of 10 milliseconds and 1 second, but also to include every intervening integration time such as 50, 100, 500, 750, and all sub-ranges such as 100-200 milliseconds, and so forth.

Any use of the term “optionally” with respect to any element of a claim is intended to mean that the subject element is required, or alternatively, is not required. Both alternatives are intended to be within the scope of the claim.

Lymphatic disorders are conditions in which there is a deviation from or interruption of the normal structure or function of the lymph or lymph vessels. Disorders of the lymphatic system affect millions of people and include, but are not limited to: lymphedema, the most severe lymphatic disorder in which patients are often susceptible to serious life-threatening cellulite infections that if uncontrolled can spread systemically or lead to amputation (both primary and secondary forms, including, but not limited to, lymphangiomatosis, lymphangioleiomyomatosis and other mixed vascular/lymphatic malformation syndromes or conditions, such as Turner-Weber and Klippel Trenauney Syndrome and those that result from filariasis, trauma, infection or surgeries of the breast, prostate, uterus, cervix, abdomen, as well as orthopedic, cosmetic (liposuction) and other surgeries, malignant melanoma, and treatments used for both Hodgkin's and non-Hodgkin's lymphoma, radiation therapy, sports injuries, tattooing, diabetes, obesity and any physical insult to the lymphatic pathways); lymphangiogenesis (or the process of growing new lymphatic structures); the inability to control infections such as that associated with HIV/AIDS; the inability to deliver antibiotic and anti-viral medication to infected tissues and organs; inflammatory and auto-immune diseases, such as but not limited to, rheumatoid arthritis and systemic lupus erythematosis, scleroderma, Wegener's granulomatosis; lymphatic insufficiency of the internal organs; impairment of lymphatic development in the intestines, for example, leads to malabsorption, ascites (collections of fat-laden lymph within the abdominal cavity), underdevelopment from malnutrition, immune malfunction, and premature death; and pulmonary lymphangiectasia, cystic hygromas and lymphangiomas that may lead to impaired vision, swallowing and breathing. Collectively, such diseases and disorders are referred to herein as “lymphatic disorders.”

As used herein, and unless otherwise indicated, the terms “treat,” “treating,” “treatment” and “therapy” contemplate an action that occurs while a patient is suffering from lymphatic disorders that reduces the severity of one or more symptoms or effects of lymphatic disorders, or a related disease or disorder. Where the context allows, the terms “treat,” “treating,” and “treatment” also refers to actions taken toward ensuring that individuals at increased risk of lymphatic disorders are able to receive appropriate surgical and/or other medical intervention prior to onset of lymphatic disorders. As used herein, and unless otherwise indicated, the terms “prevent,” “preventing,” and “prevention” contemplate an action that occurs before a patient begins to suffer from lymphatic disorders that delays the onset of, and/or inhibits or reduces the severity of, lymphatic disorders. As used herein, and unless otherwise indicated, the terms “manage,” “managing,” and “management” encompass preventing, delaying, or reducing the severity of a recurrence of lymphatic disorders in a patient who has already suffered from such a disease or condition. The terms encompass modulating the threshold, development, and/or duration of the lymphatic disorders or changing how a patient responds to the lymphatic disorders.

As used herein, and unless otherwise specified, a “therapeutically effective amount” of a compound is an amount sufficient to provide any therapeutic benefit in the treatment or management of lymphatic disorders or to delay or minimize one or more symptoms associated with lymphatic disorders. A therapeutically effective amount of a compound means an amount of the compound, alone or in combination with one or more other therapies and/or therapeutic agents, that provides any therapeutic benefit in the treatment or management of lymphatic disorders, or related diseases or disorders. The term “therapeutically effective amount” can encompass an amount that alleviates lymphatic disorders, improves or reduces lymphatic disorders, improves overall therapy, or enhances the therapeutic efficacy of another therapeutic agent. The “therapeutically effective amount” can be identified at an earlier stage with parameters of lymphatic function as identified herein.

As used herein, and unless otherwise specified, a “prophylactically effective amount” of a compound is an amount sufficient to prevent or delay the onset of lymphatic disorders, or one or more symptoms associated with lymphatic disorders or prevent or delay its recurrence. A prophylactically effective amount of a compound means an amount of the compound, alone or in combination with one or more other treatment and/or prophylactic agent that provides a prophylactic benefit in the prevention of lymphatic disorders. The term “prophylactically effective amount” can encompass an amount that prevents lymphedema-related disorders, improves overall prophylaxis, or enhances the prophylactic efficacy of another prophylactic agent. The “prophylactically effective amount” can be prescribed at an earlier stage with parameters of lymphatic function as identified herein.

As used herein, the term “lymphatic structure(s)” refers to all or a portion of structures that make up a mammalian lymphatic system including without limitation, lymph nodes, collecting vessels, lymph trunks, lymph ducts, capillaries, or combinations thereof. The architecture of the lymphatic structures can be described by tortuosity, density, dilation, and other parameters.

In this disclosure, the use of the term “real-time” or “real time” refers to activities that take place within a minute of imaging, diagnosis, or treatment of a patient. For example, “real-time” display refers to the ability to display the image while the patient is being imaged by the imaging system. For example, in some applications “real-time” therapy assessment refers to the ability to provide feedback of a particular treatment or therapy while a patient is still undergoing the treatment (e.g., MLD).

As used herein, the term “near-infrared” refers to electromagnetic radiation at wavelengths ranging from about 750 nm to about 900 nm.

The term “functional imaging” of lymph structures refers to how the structures function in terms of update of a dye, the lymphatic flow as determined by the dye, dynamics of flow, and direction of flow of lymph and the associated materials carried by it. The function of the lymphatic structures can be described by lymph velocity, period or frequency of propulsive events, permeability, and other parameters that provide evidence of dysfunction in comparison to normal function imaged in healthy control animals or human subjects. If lymph vessels are “functional” they transport materials, and imaging methods disclosed herein describe this.

The section headings used herein are for organizational purposes only and are not to be construed as limiting the subject matter described.

Overview

The circulatory system is comprised of arteries, veins, and lymphatics. Whereas established angiography using computed tomography (CT) and magnetic resonance (MR) provide means to evaluate the arteries and veins following the administration of several milliliters of contrast material containing contrast agent at greater than millimolar concentration, there has been no prior method to conveniently assess the lymphatic structure, architecture, or function. It is difficult to access the lymphatics for administration of contrast agent and there is no currently available technique that can provide contrast with a small volumetric dose of contrast agent. The publication Rasmussen et al., “Lymphatic imaging in humans with near-infrared fluorescence,” Curr Opin Biotechnol. February 2009; 20(1):74-82 is incorporated herein by reference.

As disclosed herein, new methods and compositions are provided which now make possible the ability to functionally image the lymphatic system non-invasively for prevention, diagnosis, treatment and research of lymphatic diseases. In some embodiments, small volume doses of contrast facilitate rapid imagery of the motion of lymph flow in lymphatic vessels. In exemplary embodiments, NIR fluorescence measurement of human lymphatic function is used to diagnose and assess disorders of the lymphatic system, as well as to direct, facilitate and evaluate treatments and therapies for management of lymphatic disorders. Specifically detailed is the use of the methods and compositions to direct manual decongestive therapy in cancer survivors whose lymphatic function was impaired, and to evaluate abnormal propulsive lymphatic activity in subjects with recognized lymphovascular disorders and identify those not previously known to exist, and predict those cancer survivors who are at risk of developing lymphatic system disorders as a result of radiation and nodal staging surgery. In some embodiments these methods and compositions are also used to predict lymph node metastasis in diagnosed cancer patients by evaluating lymphatic structure and function in established cancer patients. In some embodiments, the methods and compositions are used to predict evaluate lymphatic structure and function in diabetics, the obese and those suffering from other lymphatic-related disorders.

The ability to non-invasively visualize lymphatic architecture and quantify its function within asymptomatic and symptomatic limbs of subjects with lymphatic disorders such as lymphedema, as well as within the limbs of normal subjects provides an opportunity to investigate the extremes of lymph function and dysfunction in humans. The aim of one study was to evaluate differences in lymphatic architecture and function with near-infrared fluorescence imaging and the quantification of apparent lymph velocity and propulsive frequency (or determination of the lymphatic propulsion period) in the arms or legs of normal control subjects as well as subjects clinically diagnosed with unilateral lymphedema.

In some embodiments the ability to non-invasively interrogate healthy and diseased human lymphatics in vivo using microdose injections of ICG and NIR fluorescence imaging was demonstrated. In this study, NIR fluorescence provided exquisite information on the architecture of the lymphatics and provides a much needed tool for understanding lymphovascular disease.

In the first human study presented, the lymphatics of control and clinically diagnosed unilateral lymphedema subjects were qualitatively and quantitatively compared for architectural and functional differences. Both primary and secondary lymphedema are chronic, progressive, and largely uncharacterized diseases that this study sought to capture at one time point for comparison to presumed “normal” control subjects. Thus, by examining presumed “normal” controls and clinically diagnosed lymphedema subjects, images were obtained of the key differences between normal function and dysfunction.

“Normal” Lymphatic Function.

In the normal subjects, it was unexpectedly found that the overall apparent lymph velocities in left arms and legs were faster than those on the right. The differences in velocity in the arms may be due to the anatomic drainage pathways, the right arm drains to the thoracic duct while the left and both legs drain to the subclavian vein. Additionally, in the pooled analysis, it was determined that the mean velocity in legs was significantly faster than that in arms, which may reflect normal physiology and the greater need to overcome gravity for lymphatic return of fluid from the lower extremities to the venous compartment via the subclavian vein.

Aberrant Lymphatic Function

In the unilateral arm lymphedema and control subjects studied, no pooled differences in apparent lymph velocities due to diagnosis were identified. However, a significant reduction in the propulsion periods in symptomatic and asymptomatic arms when compared to control arms was identified. Without being bound by theory or any particular mechanism, it is thought that the decrease in propulsion period in the arms of lymphedema subjects as compared to controls may reflect a mechanism to compensate for the reduced number of lymphatic vessels and/or abnormal architecture.

Without being bound by theory or any particular mechanism, it is thought that there are several potential ways in which the differences between controls and asymptomatic arms may suggest that (i) the lymphedema condition may be systemic rather than localized to the symptomatic limb and/or (ii) there may have been a predisposition to lymphedema for the ten recruited arm lymphedema subjects who encountered symptoms after cancer surgery.

In the leg lymphedema and control subjects studied, statistically significant differences in the pooled apparent velocities or propulsion periods between control and both the asymptomatic and symptomatic legs of lymphedema subjects was found. However, there was no significant difference in the apparent velocities or the propulsion periods between symptomatic and asymptomatic legs. Without being bound by theory or any particular mechanism, it is thought that decreased velocity in the legs of lymphedema subjects as compared to controls suggests that velocity may slow with the progression of disease. The observed trend of decreased propulsion frequency (or increased period) coupled with a decrease in velocity may result in a net reduction of lymph transport and a lack of a compensatory mechanism to alleviate the fluid imbalance. Although statistical differences in propulsion periods and velocities exist between control and lymphedema subjects, these differences alone were too subtle for classifying disease. Without being bound by theory or any particular mechanism, applicants envision that this lack of classifiable differences seems to indicate that despite the disease status, when a lymphatic vessel is functional its velocity and period remain similar. However, more propulsion events were consistently observed in the asymptomatic than the symptomatic limbs.

Lymphatic Architecture

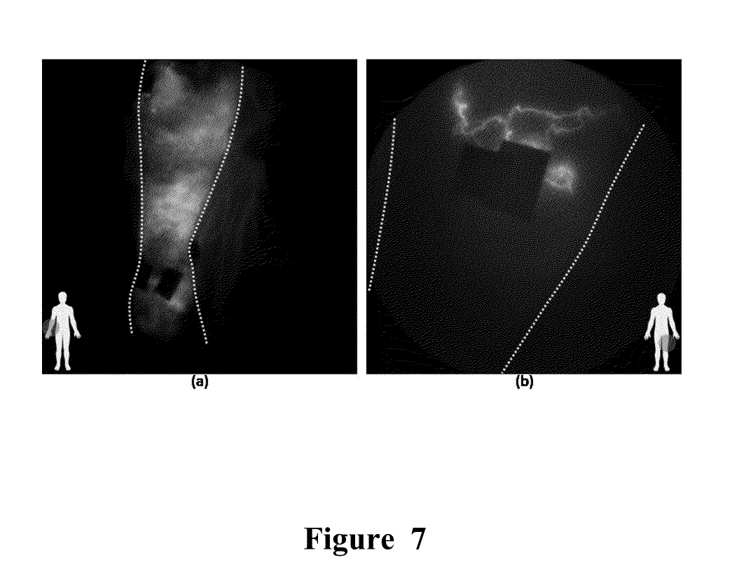

The architectural changes imaged in lymphedema subjects were striking They could be quantified in terms of tortuosity, vessel density, permeability, etc., not generally seen in the control subjects, making them potential diagnostic indicators of lymphatic disorders. The extravascular accumulation of dye, especially near the injection sites (FIG. 7a), seen in lymphedema subjects may be attributed to result from failure of initial lymphatic collection. The networks of fluorescent dermal lymphatics (FIG. 7b) may result from lymphangiogenesis that may be a normal physiological response to compensate for fluid imbalances or may be aberrant lymphangiogenesis which may possibly be contributory to the disease. The architectural features, extravascular dye accumulation and fluorescent capillary networks were only seen in two limbs of control subjects, but were observed in all symptomatic and 7 of 20 asymptomatic limbs of lymphedema subjects. Of the seven subjects with these features on the asymptomatic limb, two were diagnosed with primary lymphedema (no known trauma prior to onset), two developed lymphedema after insect bites, and three (two with arm and one with leg involvement) developed lymphedema after surgery. Without being bound by theory or any particular mechanism, it is thought that the presence of these features in asymptomatic arms may be indicative of a genetic predisposition towards the development of lymphatic disorders that was triggered by the assault on the lymphatics. Alternatively, their presence could indicate a systemic interrelationship within the lymphatics which triggers degradation of lymphatic system after another portion has been assaulted. Tortuous vessels are prevalent in both symptomatic and asymptomatic limbs of lymphedema subjects and were also visualized in five control subjects. In the control subjects, based upon the subjects' personal account of prior medical history as noted in Table 3, tortuous vessels were associated with past injury or trauma in the legs.

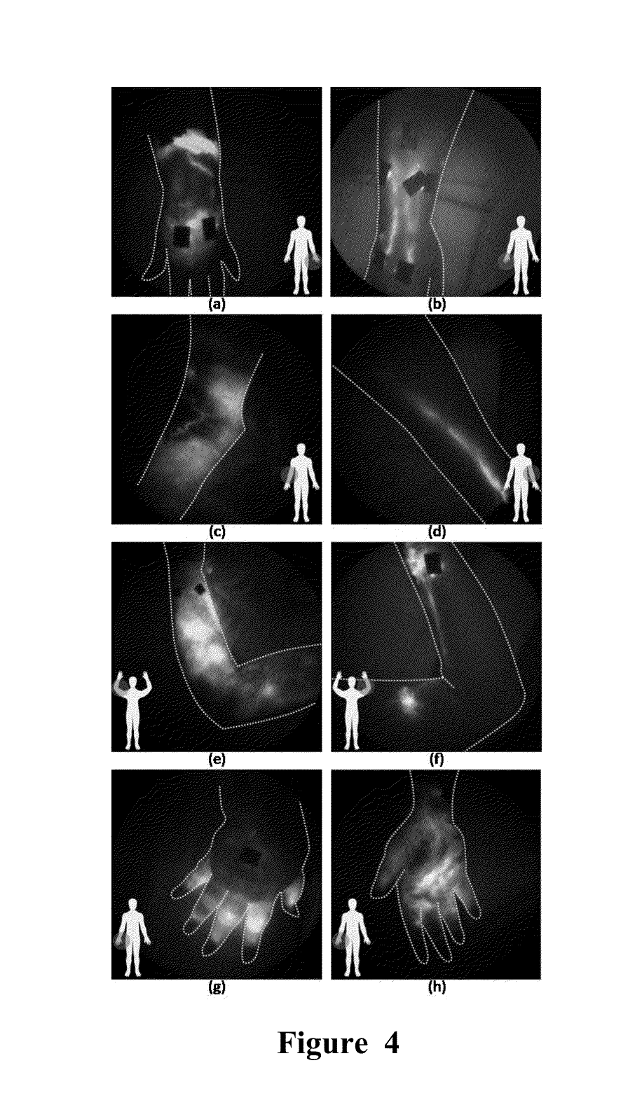

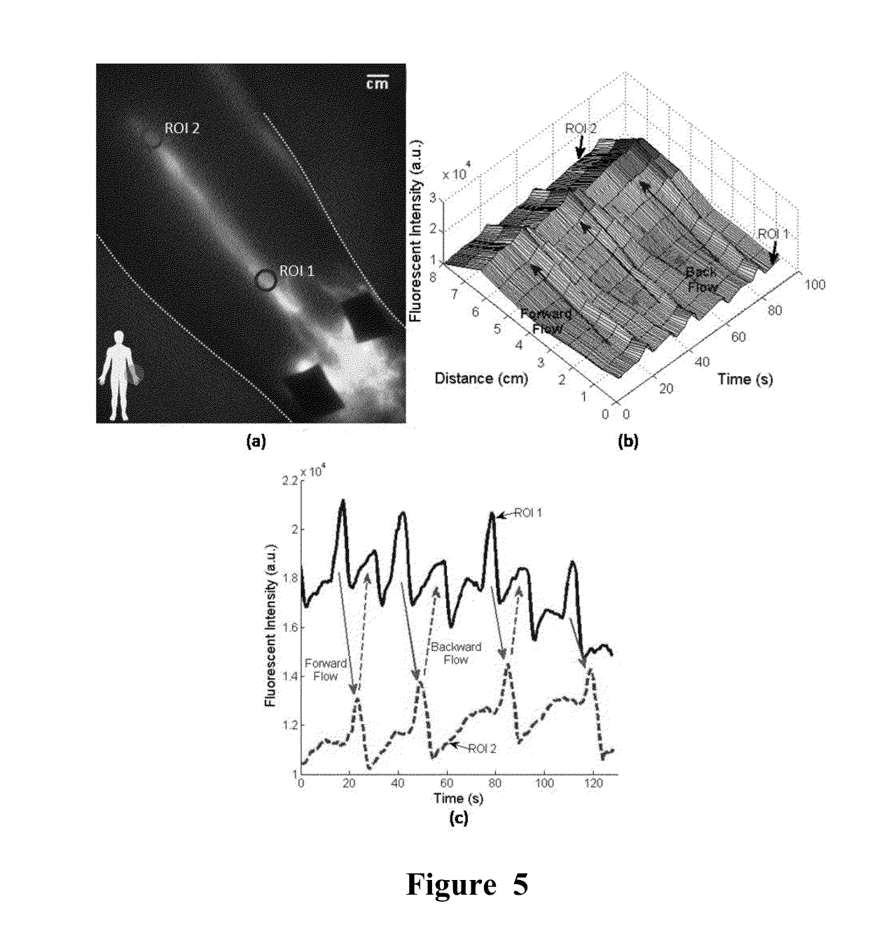

It was observed that lymphatic reflux that could be attributed to lymph valvular insufficiency in the legs of three control subjects, in one asymptomatic arm of a breast cancer lymphedema subject who had bilateral mastectomies and fluorescent lymphatic capillary networks (FIGS. 4(f) and 5), and in one symptomatic arm of another breast cancer lymphedema subject.

In conclusion, the first human study demonstrated the ability to non-invasively interrogate healthy and diseased human lymphatics in vivo using microdose injections of ICG and NIR fluorescence imaging. NIR fluorescence provided exquisite information on the architecture of the lymphatics and may provide a much needed tool for understanding lymphovascular disease.

The second human study described below demonstrates the potential of NIR fluorescence imaging to aid in diagnosing and phenotyping lymphatic disease, for pre-surgical determination of patient susceptibility to post-surgical lymphatic complications, and for the enhancement of our fundamental understanding of the lymphatic system.

Decongestive Therapy

Lymphedema is a chronic and incurable disease in which management is critical for controlling the condition. The accepted method to manage lymphedema is through the use of a non-surgical and non-pharmacological technique called complete decongestive therapy (CDT), which includes manual lymph drainage (MLD), compression bandaging, therapeutic exercise, and meticulous skin care. MLD, one of the most often used treatments for lymphedema, is hypothesized to stimulate the lymphatic contractile function and promote the clearance of lymph fluid from the affected area. MLD consists of a massage-like technique that is performed for 30-60 minutes to first stimulate lymphatic drainage from receiving lymph node basins, and then presumably stimulate contractile or “pumping” function of the superficial (epifascial) lymphatic system for subsequent drainage. Response to MLD is usually measured indirectly through reduction of limb volume using a number of accepted and experimental methods over a period of weeks to months. While CDT response rates using limb volumetric measurements are reported to be 67.7% for lower extremity and 59.1% for upper extremity LE subjects over a management period of 12 months, there remains no method (i) to predict who will respond to CDT or (ii) to measure whether contractile or “pumping” function is indeed enhanced by MLD. As some lymphedema patients do not benefit from MLD, its effectiveness remains controversial. There is no existing method currently available to immediately evaluate efficacy of MLD. A diagnostic method is disclosed herein for use as a tool in predicting a patient's benefit from MLD. A prognostic method is also provided for use as a tool to improve patient compliance, which is the leading cause of failure of MLD.

Lymphoscintigraphy is the currently accepted imaging approach for diagnosis of lymphatic dysfunction through quantifying the transit time of radionuclide transport from a distal injection site to the draining lymph node basins, its clearance from the injection site, or its accumulation in draining lymph nodes (See, for example, A, Shin W S, Strauss H W, Rockson S. The third circulation: radionuclide lymphoscintigraphy in the evaluation of lymphedema. J Nucl Med. January 2003; 44(1):43-57). Insufficient spatial and temporal resolutions can limit the lymphatic architectural and functional information that may be needed to assess response to lymphedema management. However, the inability to conduct pre- and post-MLD lymphoscintigraphy in a single therapy session does not enable efficient evaluation of lymphedema treatment.

A near-infrared (NIR) fluorescence imaging technique that has tenths of second temporal resolution to visualize the active contractile function and architecture of human lymphatics is described in U.S. Pat. Nos. 5,865,754; 7,054,002; 7,328,059; and US Patent Application Publication Nos: 2007/0286468; 2008/0056999; 2008/0064954; and 2008/0175790. This technique is akin to lymphoscintigraphy except that it employs a fluorescent contrast agent rather than a radionuclide and requires tissue surface illumination with excitation light. Because a fluorophore can be repeatedly excited to provide significant photon count rates, human imaging can be accomplished using microdose administration of fluorescent agents, thus alleviating the injection of substantial volumes of contrast agent as is performed in emerging MR and CT techniques.

In a second clinical study NIR fluorescence imaging was used to evaluate the proper course of treatment for individual patients and quantitatively evaluate the responses of active contractile pumping and apparent lymph velocity pre- and post-MLD in normal control subjects and in persons clinically diagnosed with Grade I or II unilateral, upper or lower extremity lyphedema.

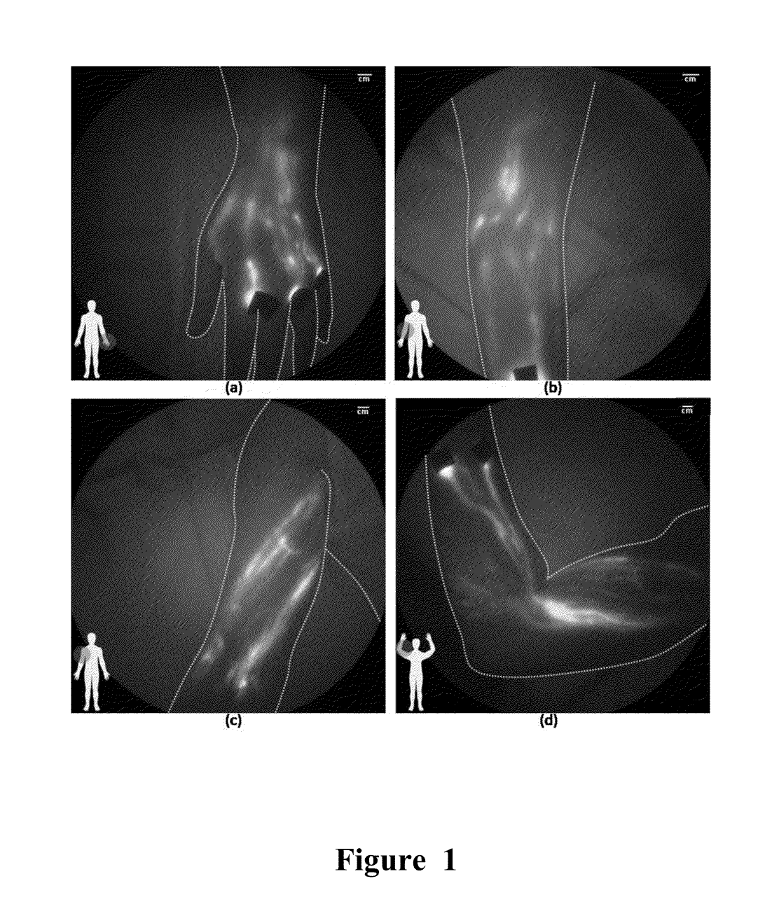

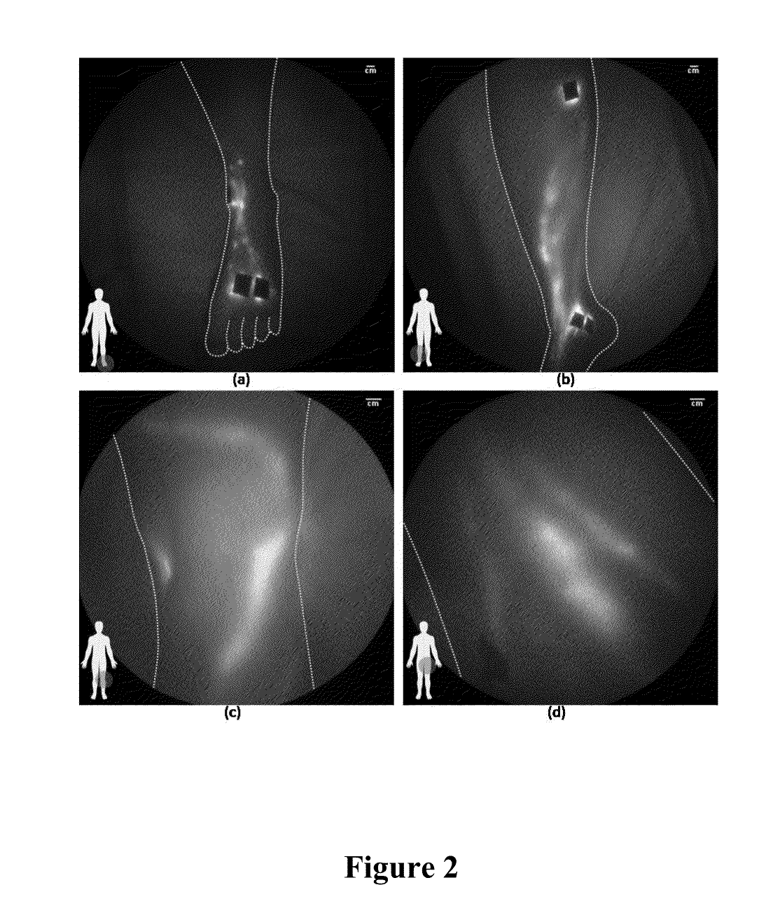

Although lymphoscintigraphy is currently the standard for assessing lymphatic function, the presently disclosed methods and compositions allow real-time NIR fluorescent imaging that not only visualizes lymphatic structures, as shown in FIGS. 1 and 2, but also determines and visualizes actual function by measuring apparent lymph velocity and propulsion period as it happens using extremely low doses of fluorescent agents. Before this study, there has been no immediate evidence of the benefit of improved lymphatic in response to MLD. To assess the effect of MLD to the lymphatics the differences of lymphatic architecture in different diagnosed (symptomatic, asymptomatic, and normal control) limbs and their functions in response to MLD was determined.

The images shown in FIGS. 1 and 2, and those obtained in supplemental videos illustrates that lymphatic architecture varies among lymphedema subjects and even among different areas on the same limb. Depending upon the severity of disease, the numbers of functional lymphatic vessels can vary within different regions of the afflicted limb. By directing lymphatic drainage towards existing functional lymphatic structures, improved responses to MLD results. Functioning lymphatic vessels that transport lymph towards major lymph node basins may be necessary for the lymphedema patient to optimally benefit from MLD. Upon using NIR fluorescence imaging to find functioning lymphatics and to manually guide drainage towards them, more efficient MLD resulted.

In these studies, it was demonstrated that in 10 lymphedema subjects, 2 (L03 and L05) out of 5 symptomatic arms and 3 (L07, L09 and L10) out of 5 symptomatic legs showed improved lymphatic function in terms of increased apparent lymph velocity and/or reduced propulsion period following MLD, suggesting MLD was a viable treatment for lymphedema in these subjects.

Stimulation of contractile function in the symptomatic limbs was not as successful as that obtained in control limbs. Without being bound by theory or any particular mechanism, it is thought that this could be due to the lack of organized lymphatic networks in lymphedema subjects. Capillary networks and tortuous vessels in lymphedema presumably result in high resistance lymph drainage pathways that impede the lymph flow even under MLD stimulation. These high resistance drainage pathways could prevent interstitial fluids from entering collecting vessels resulting in the extravascular ICG-laden lymphatic fluid that was commonly seen in symptomatic limbs in these studies. The response of the lymphatic parameters, velocity and period to MLD, in asymptomatic limbs was also reduced in comparison to the control limbs. This result is supported by the abnormal lymphatic architecture observed in asymptomatic limbs and the recent evidence of a systemic or genetic predisposition for acquired lymphedema after surgery or trauma of which 2 (L08 and L09) of 10 lymphedema subjects may have. Only the control limb group showed significant decrease in propulsion period after MLD. If partial or complete loss of lymphatic contractile function is associated with progressive lymphedema, then the stimulation with MLD would be expected to cause a diminished impact on the frequency of lymph propulsion in lymphedema subjects as compared to normal control subjects. The present study demonstrates that the response to MLD in the subjects with varying etiologies or stages of disease can be identified and characterized. Protocols to acquire sufficient data for evaluating differences in pre- and post-MLD within individual patients could lead to personalized care of lymphedema patients.

In this study, NIR fluorescence imaging was used to quantitatively assess the improved lymphatic propulsion and transport following MLD due to the temporal resolution that enables visualization of contractile function, and the ability to track lymphatic parameters pre- and post-therapy within the same session, and unique quantification of apparent lymphatic velocity and propulsion period owing to contractile lymphatic function.

In some embodiments, the imaging conjugate will also be dual labeled with a radio-isotope in order to combine imaging through nuclear approaches and be made into a unique cyclic structure and optimized for binding affinity and pharmacokinetics. Such agents can be administered by any number of methods known to those of ordinary skill in the art including, but not limited to, oral administration, inhalation, subcutaneous (sub-q), intravenous (I.V.), intraperitoneal (I.P.), intramuscular (I.M.), or intrathecal injection, or as described in greater detail below.

In some embodiments the methods and compositions described herein can be used alone or in combination with other techniques, to diagnose access and monitor and direct therapy of lymphatic disorders.

### EXAMPLES

### Example 1

### Architecture and Quantification of Lymphatic Function in Subjects with Lymphedema and in Controls

Study Design

The protocols used for the two clinical studies presented were approved by the United States' Food and Drug Administration under combinational exploratory investigational new drug application 76,920 for the off-label use of ICG as a NIR fluorescent contrast agent. These studies were approved by the Institutional Review Board at Baylor College of Medicine in Houston, Tex. where the studies was conducted, and data were analyzed under approval of the Institutional Review Board at the University of Texas Health Science Center at Houston, Tex. where the conclusions of the study were made.

In the first study, twenty-four normal volunteers and twenty subjects diagnosed with Stage I or II unilateral lymphedema were recruited. Demographic data for all 48 subjects who were imaged are presented in Table 1. The demographics, dosage, and disease etiology for each subject are shown in Table 2. Persons weighing more than 400 pounds (weight limit of bed) or with known sensitivity to iodine were excluded from the subject as were minors and pregnant and nursing women. Half of the lymphedema subjects had been clinically diagnosed with unilateral leg lymphedema and half with unilateral arm lymphedema. Half of the normal volunteers and diseased subjects received intradermal injections of ICG in both arms and the remainder in both legs. The lymphedema subjects received injections in both symptomatic and asymptomatic bilateral limbs. Immediately after injection, all volunteers were imaged with a custom-built NIR fluorescence imaging system. Total imaging time varied from subject to subject, but was typically two hours. As part of the study, half the subjects underwent manual lymphatic drainage after 30-60 minutes of imaging. only the pre-massage data is reported, with the manual lymphatic drainage results being described in detail below Subjects were supine during imaging. Vitals were monitored for two hours after injections and follow-up calls were made 24 and 48 hours later. No adverse events were associated with the drug or device in this feasibility study. Effects of gender, age, weight, and ethnicity were not examined in this feasibility study.

Injection of Contrast Agent

ICG is a tricarbocyanine dye that is approved for hepatic and ophthalmology applications. It is typically administered systemically in adults at total doses not exceeding 2 mg/kg. With a normal biological half-time of 2-4 min, being taken up by the liver and secreted into the bile, ICG has been used safely for more than forty years and can be excited between 760 and 785 nm and the emitted fluorescent signal imaged between 820 and 840 nm. In addition to ICG, those of skill in the art would readily recognize that alternative contrast agents might be used in some of the described embodiments. Such contrast agents include, compounds, such as organic dyes that exhibit fluorescence at near-infrared wavelengths when exposed to excitation light. Examples of such contrast agents which may be used in conjunction with the one or more fluorophore binding moieties to pertinent molecules associated with lymphatic function and lymphangiogenesis (e.g., α₉β₁), include, but are not limited to, indocyanin green (ICG) and other tricarbocyanine dyes, bis(carbocyanine)dyes, dicarbocyanine dyes, indol-containing dyes, polymethine dyes, acridines, anthraquinones, benzimidazols, indolenines, napthalimides, oxazines, oxonols, polyenes, porphins, squaraines, styryls, thiazols, xanthins and other NIR contrast agents known to those of skill in the art, such as IR-783 and IRDye® 800CW or combinations thereof. These exemplary contrast agents may be used in conjunction with the one or more binding moieties that bind to pertinent molecules associated with lymphatic function, the composition of lymph, or structural features of new lymph vessels formed during lymphangiogenesis, including without limitation, α₉β₁.

For these studies, ICG was reconstituted in water and subsequently diluted with saline to achieve a concentration of 0.25 mg/ml. Each subject received up to 16 intradermal injections of 0.1 ml diluted ICG for a maximum total dose of 400 μg. Injection sites varied owing to areas of fibrosis in lymphedema subjects. In the arms, four injections were administered between the digits of each hand for a total dose of 200 μg of ICG, or when fibrosis made interdigit injection difficult, a total of six injections were made on each arm, typically with two on the top of the hand, two on the medial forearm, and two on the lateral forearm for a total dose of 300 μg ICG. Generally, eight injections were administered on each leg, (two on top of the foot, two on the medial ankle, one on the heel, two on the calf, and one on the thigh) for a total dose of 400 μg ICG. One lymphedema leg subject declined additional injections after two injections were administered for a total dose of 50 μg. In all cases, the injection sites for individual subjects matched between right and left limbs. Some subjects requested the use of a topical anesthetic with lidocaine 2.5% and prilocaine 2.5% cream to lessen the sensation of needle insertion. No difference in imaging results was observed when the topical anesthetic was or was not employed. For this study, the clinically identified diseased limb of the unilateral lymphedema subject are referred to as symptomatic, the clinically unaffected limbs are referred to as asymptomatic, and all limbs of the controls subjects are referred to as normal or control.

Near-Infrared Fluorescence Imaging

Following intradermal injection, the injection sites were covered with black vinyl tape to prevent camera oversaturation. The location and movement of the ICG in each limb was then imaged simultaneously using two custom-built fluorescence imaging systems. Occasionally bilateral limbs were imaged with a single imaging system. Each system consists of a 785 nm excitation light source, a NIR sensitive image intensifier, and a customized charge coupled device camera outfitted with filters to collect fluorescence at 830 nm. After imaging the first eight control subjects, the original 80 mW laser diode used to provide the 785 nm excitation light source was replaced with a 500 mW laser diode to provide a broader, brighter, and more uniform illumination of the field of view resulting in sharper and more detailed fluorescent images. The emitted excitation light was attenuated using an optical diffuser such that a maximal tissue surface area of approximately 900 cm² was illuminated with less than 1.9 mW/cm². Excitation light propagated through tissues to activate the injected ICG. The generated fluorescent signal that emanated from the tissues was filtered with a 785 nm holographic notch filter (optical density ≧6) and an 830 nm bandpass filter (optical density ≧4). A 28 mm Nikkor lens (Nikon, Melville, N.Y.) was used to focus the fluorescent signal onto the photocathode of a Gen 3 image intensifier. The intensified image was acquired with a customized, 16 bit, frame transfer, CCD camera. A total acquisition time of −650 ms per image permitted near real time imaging of the lymphatics in vivo and enabled a compilation of images to create a movie of lymph flow.

Quantification of Lymphatic Function

The two dimensional fluorescent images were previewed using ImageJ (National Institutes of Health, Bethesda, Md.) to visually identify subsets of images containing active lymph propulsion as distinguished by the movement of higher fluorescent intensity ‘packets’ of lymph along lymphatic vessels. A custom MATLAB (Mathworks, Natick, Mass.) program was then used to identify regions of interest (ROI) along the length of the lymphatic vessels and to calculate the apparent distance each packet of lymph traveled between ROIs. The distance is denoted as apparent to reflect the lack of information in the third dimension. Apparent velocity was computed by dividing the apparent distance traveled by the travel time of the lymph ‘packet.’ In cases where fluorescence was observed to move distally from the local draining lymph basin, the apparent velocities were reported as negative. The period was measured as the time lapse between consecutive lymphatic propulsion events in the same ROI. In this initial study, the limb was interrogated by scanning the camera field of view across the entire limb.

Statistical Analysis

A language and environment for statistical computing, as is known in the art, was used to perform analysis of variance (ANOVA) and pairwise or Welch t-tests to determine the relationship between diagnosis (normal, symptomatic, or asymptomatic limb), limb (arm or leg), and side (right or left) and the log transforms of both velocity and period. For study wide analysis, velocity and period measurements were pooled by groups of interest (i.e., all control arm measurements vs. all symptomatic arm measurements, all arm measurements vs. all leg measurements, etc.). In addition, t-tests were done to determine the effect of side (right or left for control or symptomatic or asymptomatic for lymphedema subjects) in the velocities and periods of each individual subject. Because of their scarcity, negative velocities due to lymphatic reflux were excluded from the statistical analysis. The Holm test was used to correct the p-value when doing group-wise comparisons. For all analyses, the tested effect was determined to be significant when the p-value was less than 0.05, when no pulses were observed yielding a velocity of zero in one of the subject's limbs, or when only active backward propulsion was observed. The reported means and standard deviations are retransformed and presented as mean (lower bound, upper bound).

Results

Tables 3 and 4 summarize the apparent lymph velocities, propulsion periods, and pertinent comments for individual control (Table 3) and lymphedema subjects (Table 4). For the pooled measurements of all 44 subjects in the study, ANOVA indicates that diagnosis, limb, and side impact both velocity (p=0.0077, p=0.012, and p=2.8×10⁻⁷ respectively) and period (p=0.016, p=1.6×10⁻⁹, and p=8.8×10⁻⁶ respectively). Architecture and quantification of lymphatic function in control limbs as well as in the limbs of subjects with clinically diagnosed unilateral lymphedema is described. Specific cases reference the subject IDs found in Tables 2-4.

Lymphatic Function and Architecture in Control Arms

While the number and anatomical map of lymphatic vessels varied between control subjects, the lymphatic structure generally consisted of well-defined channels as illustrated in FIG. 1. FIG. 1a shows the web of lymphatic vessels in the dorsal left hand after four interdigital injections of ICG; FIG. 1b shows the lymphatic vasculature in the medial right arm; and FIGS. 1c and 1d show the lymph vessels in the proximal and entire right arm respectively. By analyzing video, active propulsive lymph flow towards the axilla was observed in all control arms. However, not every fluorescent vessel actively propelled lymph.

For the twelve pairs of control arms investigated, only one subject exhibited statistically different velocities in right and left arms, with the faster apparent velocities on the left. However, for the pooled control arm data, the difference in velocities in the left (0.8(0.5, 1.2) cm/s) and right (0.7(0.5, 1.0) cm/s) arms was significant (p=8.2×10⁻⁵) though subtle. Only one of the twelve pairs of control arms exhibited significantly different propulsion periods. For the pooled control arm data, the difference in propulsion periods, 37.4(18.1, 77.2) s and 37.9(19.1, 75.6) s in the left and right arms respectively, was not significant.

Lymphatic Architecture and Function in Control Legs

Similar to arms, the lymphatic architecture in the legs also varied between control subjects, but generally consisted of well defined structures as illustrated in FIG. 2. FIG. 2a illustrates the web of lymphatics in the dorsum of the left foot after two injections of ICG approximately one inch above the digits. Video visualization of lymphatic flow in the foot was obtained. In FIG. 2b the lymphatics located in the medial right leg are shown; in FIG. 2c two lymphatic vessels are visible in the right patellar fossa; and FIG. 2d shows deep lymphatics vessels draining towards the inguinal lymph node basin in the left leg. Lymphatic vessels located deep within the leg appear diffuse owing to the scatter of light from intervening tissues. In five subjects (indicated in Table 3), tortuous lymphatic vessels were also seen and in some cases seemingly corresponded with a subject's description of prior injury such as chemical burn or sprain. In one control limb a small area of extravascular dye was observed near an injection site while several smaller vessels were observed radiating from an injection site another control limb. Lymphatic reflux or passive backflow of a packet of lymph towards the feet was observed in three control subjects.

There were no significant differences found between the left and right velocities or periods in the legs of individual subjects (see Table 3). However, analysis of the pooled leg data indicated a subtle but significant (p=0.0059) difference between the overall apparent velocities in the left (0.8(0.5, 1.5) cm/s) and right (0.7(0.4, 1.3) cm/s) legs, while the difference in the propulsion periods (left: 40.6(18.2, 90.2) s, right: 40.4(20.1, 81.5) s) is not significant.

Analysis on the pooled apparent velocities in the arms (0.7(0.5, 1.1) cm/s) and legs (0.8(0.5, 1.4) cm/s) indicates that while subtle, the difference between the limbs was significant (p=5.7×10⁻⁵). However the difference in the overall propulsion periods between arms (37.7(18.7, 76.2) s) and legs (40.5(19.0, 86.4) s) was not significant.

Drainage to Lymph Nodes

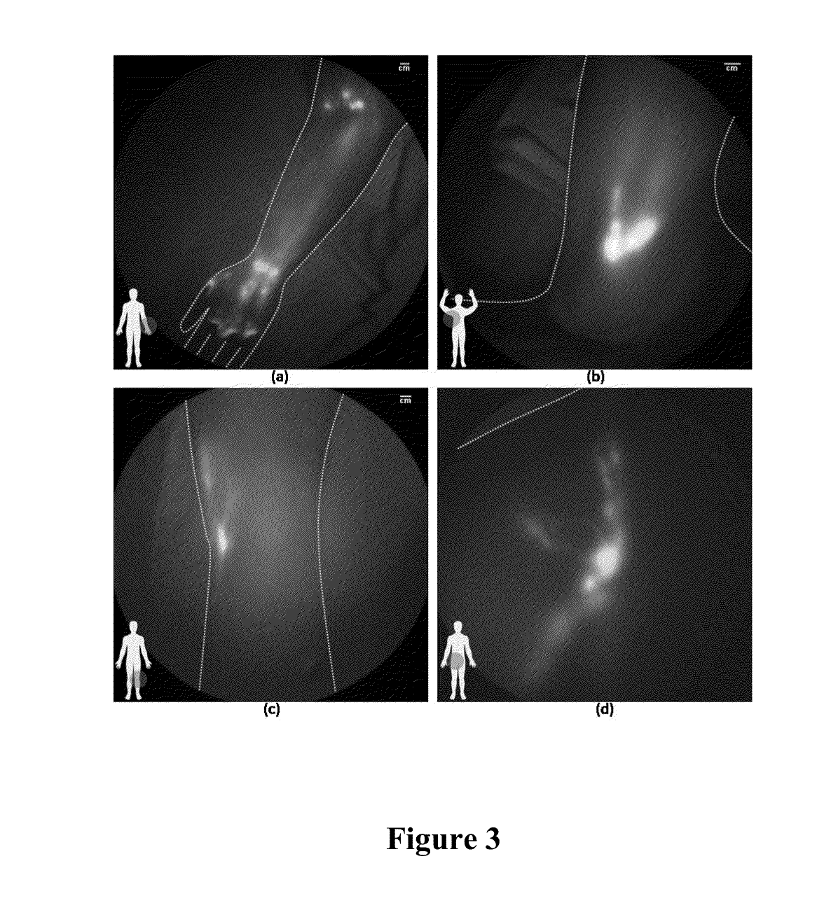

The lymph traveled to regional lymph nodal basins as shown in FIG. 3. The supraclavicular lymph nodes (not shown), cubital lymph nodes (FIG. 3a), axillary lymph node chain (FIG. 3b), popliteal lymph node (FIG. 3c), and the inguinal lymph nodes (FIG. 3d) were imaged.

Diseased Lymphatic Architecture and Function in Subjects with Arm Lymphedema

The lymphatic architecture in lymphedema subjects was markedly different than that in controls. Architectural abnormalities such as regions of diffused dye patterns arising from extravascular fluorescence and dense networks of capillary lymphatics were not typically observed in the control subjects. While tortuous vessels were observed in five of forty-eight control limbs, they were seen in nearly half of the forty asymptomatic and symptomatic limbs (legs and arms).

FIG. 4 illustrates symptomatic and asymptomatic hands and arms of typical subjects with acquired lymphedema following breast cancer treatment. FIG. 4a shows extravascular fluorescence just above and below the wrist with a conducting lymphatic channel connecting the two areas (FIG. 4a) while the asymptomatic hand has well defined lymphatic architecture (FIG. 4b). The ventral view of a symptomatic elbow exhibited extravascular fluorescence and tortuous vessels (FIG. 4c) but only a single lymphatic vessel was observed on the asymptomatic arm (FIG. 4d). The case of one subject, who underwent bilateral mastectomies nine years apart and had developed clinically diagnosed unilateral right arm lymphedema about one year prior to the second (left breast) mastectomy, is noteworthy. Her right, symptomatic arm had extravascular fluorescence throughout the entire arm (FIG. 4e) while the left, clinically unremarkable, asymptomatic arm had two areas of fluorescent lymphatic capillaries with tortuous vessels near the wrist and upper arm (FIG. 4f) as well as lymphatic reflux in the main lymphatic channel just above the wrist. Another noteworthy case is that of a post-mastectomy subject who had active backward propulsion of lymph into her hand and fingers (FIG. 4g). To relieve the subsequent fluid pressure, a spontaneous fistula or “weep hole” had developed in the palm of her hand (FIG. 4h) from which, as reported by the subject, fluid leaked periodically. Manual lymphatic drainage therapy did not mitigate her lymphedema.

FIG. 5a shows a closer view of the lymphatic vessel with reflux in the asymptomatic limb previously shown in FIG. 5f. This was also visualized in a video recording of the lymphatic reflux. FIG. 5b shows a three dimensional plot of the fluorescent intensity as a function of time and distance traveled along the lymphatic vessel. By tracking the appearance of “peaks” of fluorescence, the forward flow or propagation of packets of lymph in the vessel and the subsequent reflux of a portion of each packet can be identified. FIG. 5c shows the fluorescent intensity profiles for two ROIs (FIG. 5a) located 8 cm apart. While active flow was observed in every asymptomatic arm of persons with lymphedema, only three propulsion events were observed in two asymptomatic arms while two or less propulsion events were seen in six symptomatic limbs (see Table 4). On average, 60±76 more pulses were seen in the asymptomatic arms than the contralateral symptomatic limbs. Passive lymph reflux was observed in the arms of two subjects with lymphedema.

Of the ten arm lymphedema cases investigated, two subjects had significantly faster flow in the symptomatic limb, one had significantly faster flow in the asymptomatic limb, and in one symptomatic limb only active backward propulsion was seen (see Table 4).

From the ANOVA on the pooled arm data, no statistical evidence of a relationship between lymphedema diagnosis and the apparent velocities in the arms was identified. The average apparent velocities were 0.7(0.5, 1.1) cm/s (control), 0.7(0.4, 1.1) cm/s (asymptomatic), and 0.7(0.4, 1.4) cm/s (symptomatic). However, significant differences were found in the pooled propulsion periods in the control (37.7(18.7, 76.2) s) and asymptomatic (30.5(14.3, 64.9) s) arms (p=0.0032), and the control and symptomatic (28.3(15.8, 50.9) s) arms (p=0.043), but not in the asymptomatic and symptomatic arms.

Lymphatic Architecture and Function in Subjects with Leg Lymphedema

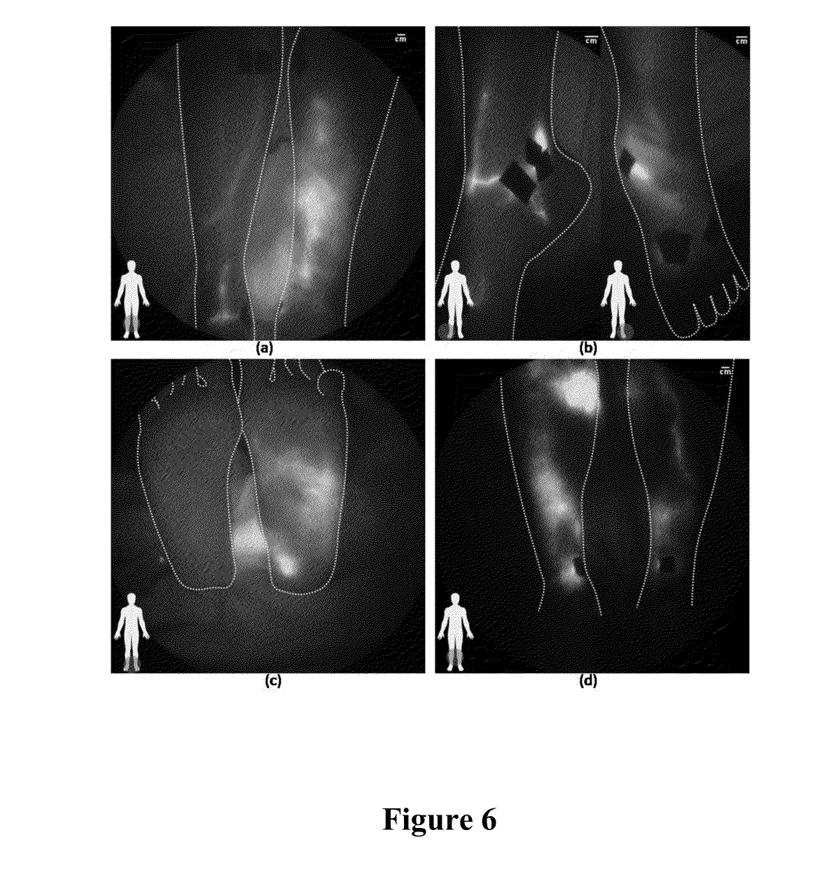

Many of the lymphatic architectural features seen in symptomatic and asymptomatic arms also occurred in leg lymphedema. As an example FIG. 6(a) indicates extensive extravascular fluorescence over much of the symptomatic leg while the asymptomatic leg appears to have few but well-defined lymphatics vessels. A closer examination of the ankles of the same subject (FIG. 6b) reveals abnormal tortuous lymphatics draining from the injection site toward the bottom of the asymptomatic foot. However, no fluorescence is visible in the sole of the asymptomatic foot while extravascular fluorescence is observed in the sole of the symptomatic foot (FIG. 6c). FIG. 6d shows both legs of a subject who was clinically diagnosed with unilateral lymphedema of the right leg after right inguinal lymph node dissection. While the asymptomatic leg shows some lymphatic structure, both legs have extensive regions of extravascular fluorescence. Of the ten unilateral leg lymphedema cases investigated, two subjects had no observed lymph flow in the symptomatic limb (See Table 4). Statistical analyses show subtle but significant differences between the apparent velocities in the control (0.8(0.5, 1.4) cm/s) and asymptomatic (0.7(0.4, 1.3) cm/s) legs (p=0.015) and the control and symptomatic (0.7(0.4, 1.2) cm/s) legs (p=0.015) but not the asymptomatic and symptomatic legs. Similarly, significant differences in propulsion period were found between the control (40.5(19.0, 86.4) s) and asymptomatic (52.5(27.0, 102.1) s) legs (p=0.0068), the control and symptomatic (58.6(29.3, 117.4) s) legs (p=0.00045), but not between the asymptomatic and symptomatic legs. On average, 6±13 more pulses were seen in the asymptomatic legs than the contralateral symptomatic legs, with only two subjects exhibiting more pulses in the symptomatic legs.

### Example 2

### Identification of Abnormal Lymphatic Architecture in Different Diagnosed Limbs and Assessing the Effect of MLD

Study Design/Materials and Methods

The protocol used for the second study was also approved under combinational exploratory (Phase 0) investigational new drug (eIND) application 76,920 for the off-label use of indocyanine green (ICG) as a NIR fluorescent contrast agent. The HIPPA-compliant studies were approved by the Institutional Review Board (IRB) at Baylor College of Medicine in Houston, Tex. where the trials were conducted and by the IRB at The University of Texas Health Science Center where the data was evaluated. Twelve normal volunteers and 10 subjects clinically diagnosed with Grade I or II unilateral LE participated and provided informed consent. Table 5 provides pertinent demographic and clinically relevant information for the 22 subjects in the study. The control subjects consisted of 2 males and 10 females, aged 22-59 years. The LE subjects were all female, aged 18-68 years with half experiencing lower extremity LE and the other half upper extremity LE. The injection sites were cleaned with a surgical scrub, followed by alcohol. Immediately after intradermal injection of imaging contrast agent using a 27 gauge needle, NIR fluorescence images were simultaneously acquired from both the LE and contralateral limbs of LE subjects. For control subjects, bilateral arms were imaged simultaneously in 6 subjects, and bilateral legs were imaged in the other 6 subjects. MLD was performed 30 to 60 minutes after the start of imaging followed by another 30 to 60 minutes of imaging. Vital signs were monitored for 2 hours after fluorescent agent administration and follow-up phone calls to assess subjects' condition were made 24 and 48 hours later. Owing to their susceptibility to infection, LE subjects were provided a prescription for antibiotic in the event that erythema and/or edema occurred at the injection site or if the subjects experienced pyrexia. No adverse events were associated with the imaging agent or device in this trial.

Contrast Agent

The imaging contrast agent used was indocyanine green (ICG: IC-Green, AKORN Pharmaceuticals, Buffalo Grove, Ill., or Indocyanine green, USP, The Medicine Shoppe Pharmacy, Kingsport, Tenn.) that fluoresces in NIR light. ICG was reconstituted in water and diluted in saline to a concentration of 0.25 mg/ml. Each subject received up to 16 intradermal injections of 0.1 ml diluted ICG for a maximum total dose of 400 μg. The agent was injected in bilateral limbs to be imaged, either arms or legs. Injection sites varied owing to areas of fibrosis in LE subjects, but injection sites were symmetrical on contralateral limbs. The maximum number of injections was 6 in each arm and 8 in each leg. Generally, there were 4 injections between the digits on each hand and 2 on each forearm. When interdigit injection was impractical due to tissue fibrosis in LE subjects or to match injections of control with LE subjects, 2 injections were made on the dorsum of the hand, 2 on the medial forearm, and 2 on the lateral forearm. In legs 2 injections were made in the dorsum of the foot, 2 on the medial ankle, 1 on the heel, 2 on the calf, and 1 on the thigh. In most cases, the maximum numbers of injection were performed. Subjects were offered the option to receive a topical anesthetic (lidocaine 2.5% and prilocaine 2.5% cream, such as Emla Cream, AstraZeneca LP, Wilmington, Del.) at the injection site to lessen the sensation of needle injection. No difference in imaging results was observed regardless of whether topical anesthesia was employed. The symptomatic limb is defined as the limb that was diagnosed as having LE while the asymptomatic limb is the corresponding contralateral limb with no clinical symptoms of lymphatic disease.

Near-Infrared Fluorescence Imaging

Following injection of ICG, the injection sites were covered with black vinyl tape to block the high fluorescence intensity due to the high local ICG concentration so that oversaturation of camera was eliminated. The location and movement of lymphatic fluid containing ICG was imaged simultaneously in both limbs using two custom-built fluorescence imaging systems. Each system consisted of an excitation light source, a NIR sensitive image intensifier, and a customized charge coupled device (CCD) camera as described in U.S. Pat. Nos. 5,865,754; 7,054,002; 7,328,059; and US Patent Application Publications Nos: 2007/0286468; 2008/0056999, 2008/0064954; and 2008/0175790, for example. The tissue area of illumination was approximately 900 cm² with the power density of less than 1.9 mW/cm² and the fluorescence images were acquired using a 200 msec camera integration time. With the additional 400 ms due to the instrumentation overhead (CCD readout time, etc.), the total acquisition time of about 600 ms per frame permitted near real time imaging of the lymphatics in vivo and provided opportunities to visualize lymphatic structure and quantify dynamic lymphatic function. Total imaging time varied from subject to subject, however the typical total imaging time was approximately 2 hours. Subjects were supine on a bed during imaging. When the field of view changed, a grid of known dimension was placed on the subject's skin to assist in focusing the camera and provide a reference measurement of length scale for later image analysis.

Manual lymphatic drainage (MLD)

MLD was performed to bare skin by a certified LE therapist during the imaging sections; neither oils nor lotions were used during MLD therapy. MLD was performed by initially providing a gentle massage to the cervical lymph nodes for 3 minutes followed by 5 minute massages of axillary and inguinal nodes in the preparation period. In subjects with arm LE, the areas that were treated with massage were the neck followed by the axillary region on the contralateral (asymptomatic) arm and the ispilateral (symptomatic side) inguinal region. For leg LE subjects, the procedure consisted of massage at the neck followed by treatment of the contralateral inguinal nodes and ispilateral axillary lymph nodes. For control subjects following 3 minutes of massage at the neck, the 5 minute massages were performed at the bilateral axillary regions if the arms were being imaged or at the bilateral inguinal regions if the legs were being imaged.

After this preparation period, the limbs being imaged were massaged with centripetal light strokes starting at the proximal aspect of the limb following with more distal segments. For the arms, the MLD protocols for both control and LE subjects were conducted in the following order: (i) 5 minutes on the upper arm, (ii) 5 minutes on the forearm, and (iii) 5 minutes on the hand. Similarly for the legs, the order was (i) 5 minutes on the thigh, (ii) 5 minutes on the leg, and (iii) 5 minutes on the foot. Live fluorescence images were available to therapist during MLD to provide guidance of lymphatic architecture.

Quantification of Lymphatic Function

Image subsets were analyzed using a computer program developed in MATLAB (Mathworks Inc., Natick, Mass.) to assess apparent lymph velocities and periods between lymphatic propulsion events as described by described above. Briefly, lymph channels were selected from the images, and average fluorescent intensities within regions of interest (ROIs) of equivalent size were used to quantify fluorescent intensity changes as functions of lymphatic vessel length and imaging time. Lymph “packets” were located by identifying the peaks in the intensity profiles of ROIs along the vessel. By tracking the locations of each packet as a function of time, the apparent velocity of the lymphatic propulsion for each individual packet was calculated. The time lapse between two “packets” passing a ROI in a vessel was recorded as the lymphatic propulsion period. Apparent velocities and periods of lymph propulsions were used as indices for the evaluation of lymphatic function. The data collected before MLD was performed were classified as “pre-MLD” and the data after as “post-MLD” results.

Statistical Analysis

Statistical analysis of the collected apparent velocities and propulsion periods was performed using MATLAB. The distribution of apparent velocities and propulsion periods were assumed to be log-normal. Analysis of variance (ANOVA) and paired t-tests of the logarithm-transformed data were performed to determine the relationship of lymphatic functions between pre- and post-MLD for different subject diagnoses (control, symptomatic, and asymptomatic) and limbs (arm and leg). For data from each individual subject, t-tests were performed to investigate the factor of MLD on each side (“right” or “left” for control subjects, or “symptomatic” or “asymptomatic” for LE subjects). For study-wide analysis, the data were pooled by groups of interest (i.e. all asymptomatic arm data vs. all symptomatic arm data, all arm data vs. all leg data, etc.) to investigate differences between groups. For all analyses, the tested factor was determined to be significant when the p-value was less than 0.05.

Results

Table 5 summarizes the ranges of pre- and post-MLD apparent lymph velocities and propulsion periods obtained from the image analysis of the data from control and LE subjects. Subject identity (ID) numbers described in the text refer to the data listed in Table 5. The number of pulses from which velocities were computed was higher than that from which periods were computed. This occurred because only one pulse was seen during imaging in some lymphatic vessels, and the period between sequential pulses could therefore not be defined.

Characteristics of Normal Limbs of Control Subjects and Symptomatic/Asymptomatic Limbs of LE Subjects

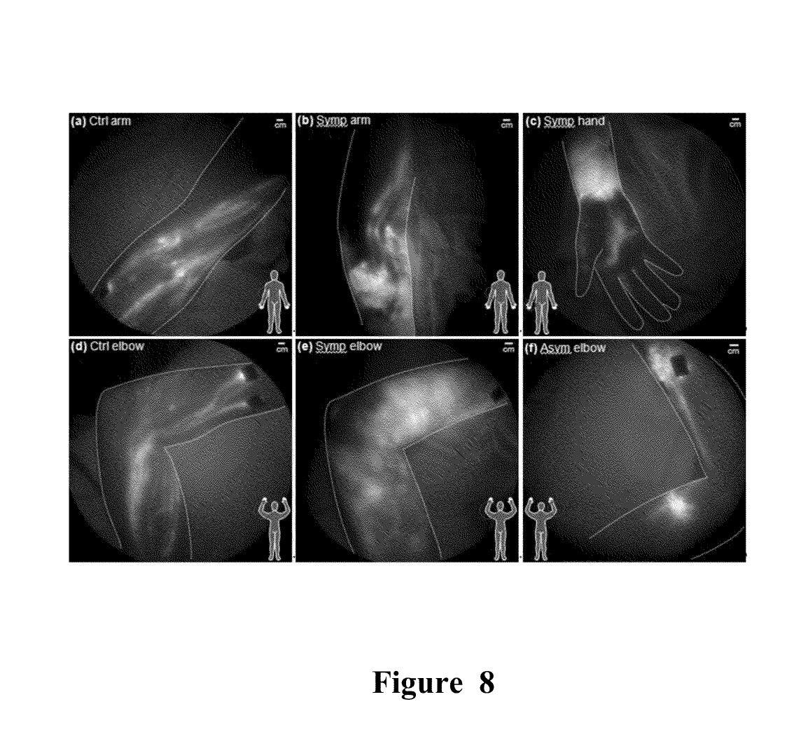

As previously observed, striking architectural differences exist between control limbs and symptomatic and asymptomatic LE limbs. As shown in FIGS. 8(a) and 8(d), the lymphatic structure in the arms of control subjects generally consists of well-defined channels that actively propel and drain lymph into the regional nodal basins. The active lymph propulsion from arm toward axillary lymph nodes during MLD were also observed in a video recording. In contrast, the lymphatic structure of symptomatic arms of LE subjects typically displays capillary networks, tortuous vessels, or extravascular lymphatic fluid leakage as shown in FIGS. 8(b) and 8(e) that may be associated with increased resistance to lymph flow. Generally, in regions within the symptomatic arms of LE subjects, distinguishable lymphatic channels that propel lymph were found in fewer numbers than in the control arms or they were not found at all. For example, subject L02 was diagnosed with Grade I LE in the right arm 8 years after radical mastectomy of the right breast and also underwent radical mastectomy of the left breast 9 years after the first mastectomy. In her symptomatic (right) arm, few lymphatic channels were found on the lateral side of the elbow as shown in FIG. 8(b) but no channels were found on the medial side as shown in FIG. 8(e). In a video showing image sequences recorded during MLD on the symptomatic arm of this subject, it was observed that typically fewer lymphatic propulsion events were seen in symptomatic arms than in asymptomatic or control arms. A total of 55 pulses were observed in 5 symptomatic arms, 129 pulses in 5 asymptomatic arms, and 798 pulses in 12 control arms, pre-MLD. In two LE cases (subjects L01 and L02), dermal backflow was observed draining toward the hand, where ICG drained from the injection site toward distal direction and filled the interstitial space of the palm as shown in FIG. 8(c).

Generally in the asymptomatic arms of LE subjects, lymphatic architecture was found to be similar to the arms of normal control subjects. However, in one case (subject L02) abnormal lymphatic patterns similar to those observed in symptomatic arms were evident. As shown in FIG. 8(f), capillary networks were observed in distinct regions of the subject's asymptomatic arm due presumably to the surgical trauma of mastectomy. The abnormal lymphatics in the asymptomatic arm of this subject may be an early indication of LE that has not yet progressed towards clinical manifestation of disease.

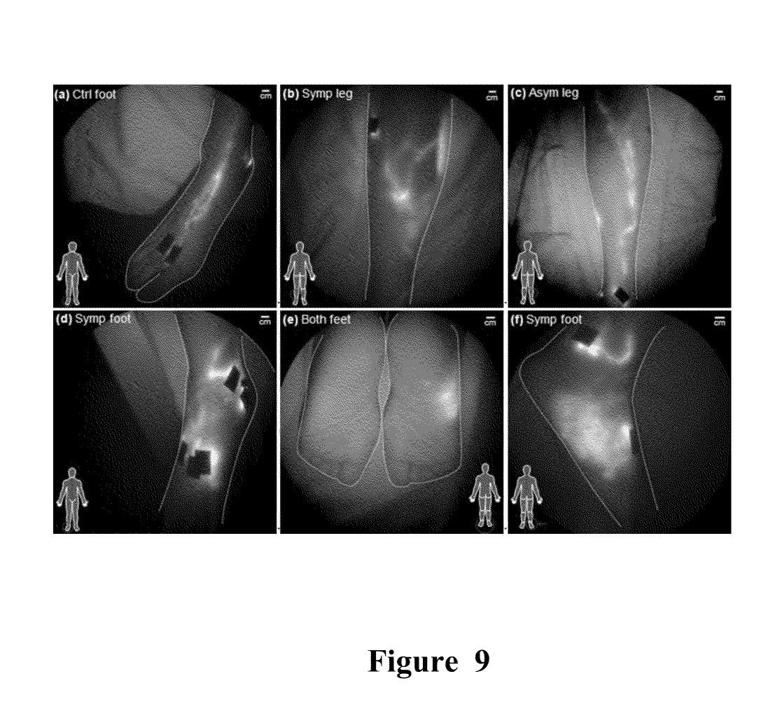

Similar findings in the control legs of normal subjects and in the symptomatic and asymptomatic legs of LE subjects were observed. FIG. 9(a) shows distinct functional lymphatic vessels typically found in the legs of a normal subject (subject N11) while capillary networks, tortuous vessels, or extravascular lymphatic fluid leakage were typically seen in symptomatic legs as observed in subjects L07 and L10 shown in FIGS. 9(b) and 9(d). A supplemental video showed the image sequences recorded during MLD on the leg in FIG. 9(d). Again, functional lymphatic channels and propulsion were seen in the symptomatic legs of some subjects (such as subject L07 as shown in FIG. 9(b); lymphatic propulsion on a video recording), while for others, no functional lymphatic vessels were observed (data not shown). In the asymptomatic legs of LE subjects, the lymphatic vessel architecture was found to be similar to the control legs in normal subjects as shown in the case of subject L07 illustrated in FIG. 9(c). However, abnormal lymphatic patterns were seen on asymptomatic legs in 4 LE subjects (L06, L08, L09, and L10), perhaps suggestive of preclinical bilateral disease rather than clinically diagnosed unilateral disease. In three symptomatic legs (subjects L06, L08 and L10), dermal backflow was seen in the foot, where ICG flowed within the lymph vessels from injection site toward the bottom of the foot and filled the interstitial space as shown in FIGS. 9(e) and 9(f).

Effect of Manual Lymph Drainage in Arms

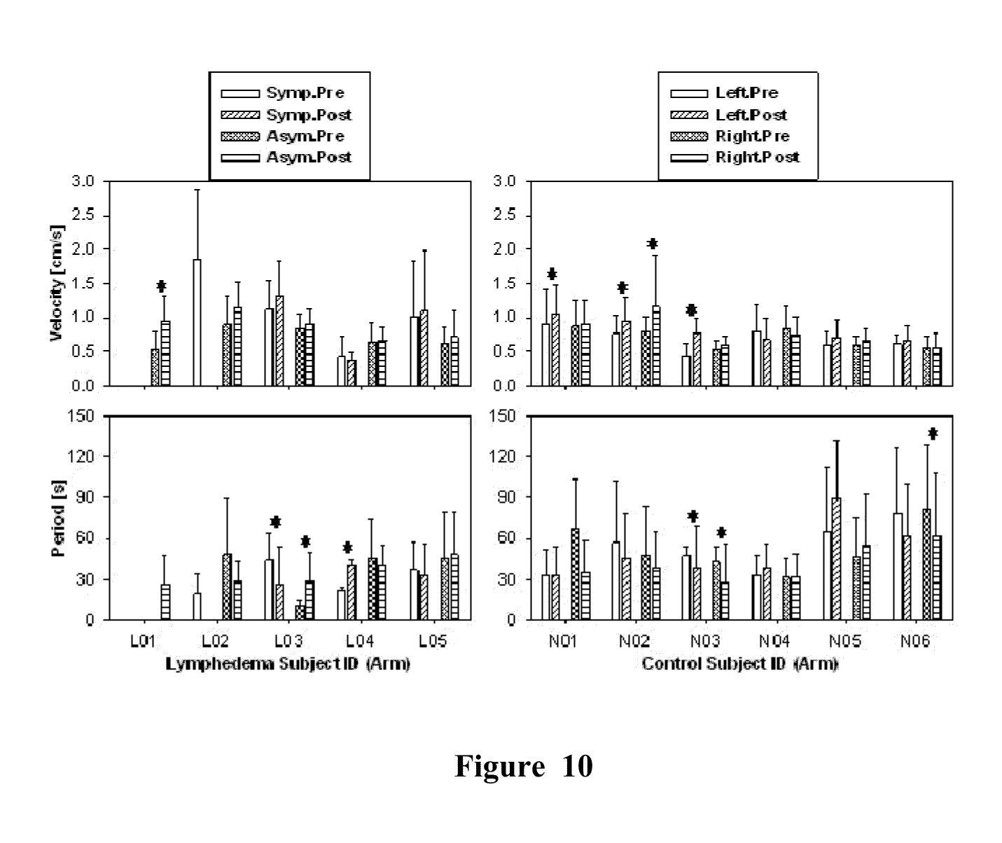

The averages of the pre- and post-MLD apparent lymph velocities and propulsion periods obtained from the symptomatic and asymptomatic arms of 5 LE subjects and the normal (both right and left) arms of 6 control subjects are shown in FIG. 10. The comparisons showing significant difference (p<0.05) between pre- and post-MLD are marked with an asterisk. None of the symptomatic arms and one asymptomatic arm showed statistically significant change in apparent lymph velocity after MLD. The asymptomatic arm of subject L03 and the symptomatic arm of subject L04 showed statistically significant increases in lymph propulsion period, while the symptomatic arm of subject L03 showed statistically significant decrease in propulsive period following MLD. Two of the 5 LE subjects (L03 and L05) experienced improved function as indicated by showing increase in apparent lymph velocity and decrease in propulsive period in symptomatic arms. After MLD, 4 of the 12 control arms showed statistically significant increase in apparent lymph velocity, and 3 of the 12 control arms showed statistically significant decrease in lymph propulsion period. With the exception of subject N04, all control arms showed a trend of increased lymph velocity after MLD. With the exception of subjects N04 and N06, all control arms showed decrease or no change in propulsion period. Because imaging times varied between subjects in this feasibility trial, no statistical correlation can be meaningfully determined for the number of “packets” of propelled lymph. However it is noteworthy that there were a total of 55 and 85 pulses observed in 5 symptomatic arms, pre- and post-MLD; while 129 and 293 pulses in 5 asymptomatic arms, pre- and post-MLD; and 798 and 802 pulses in 12 control arms, pre- and post-MLD. No propulsion was found in the symptomatic arms of both subjects L01 and L02, implying ineffectiveness of MLD. The dermal backflow seen in their hands might be one of the reasons for the failure of MLD. It is noteworthy that subject L01 was clinically unresponsive to intensive MLD and bandaging conducted over a period of 6 weeks.

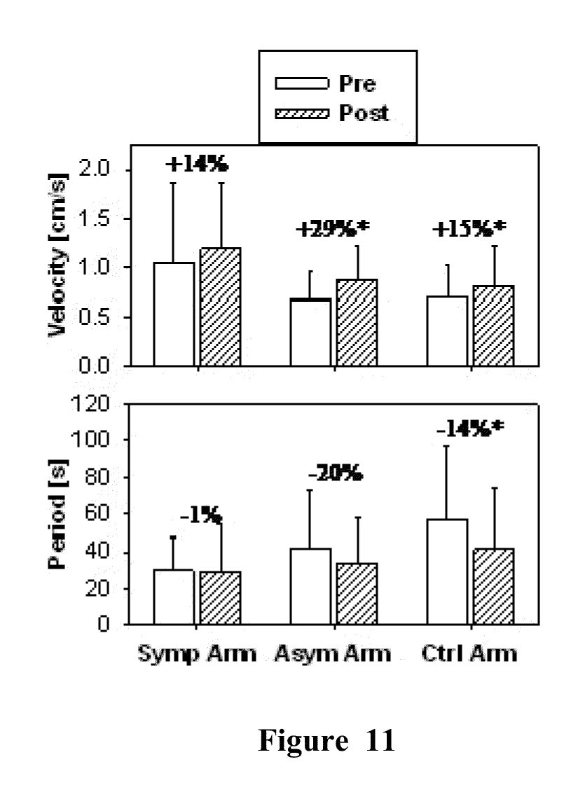

The statistical results of the pooled data grouped into symptomatic, asymptomatic, and control arms are shown in FIG. 11. The average apparent lymph velocities and propulsion periods are plotted for each group with the percentages of changes from pre- to post-MLD indicated. The lymph velocities increased for all groups after MLD, and the increases were statistically significant in asymptomatic and control arms (see Table 6). The periods decreased for all groups, and the decrease for control arm group was statistically significant.

Effect of Manual Lymph Drainage in Legs

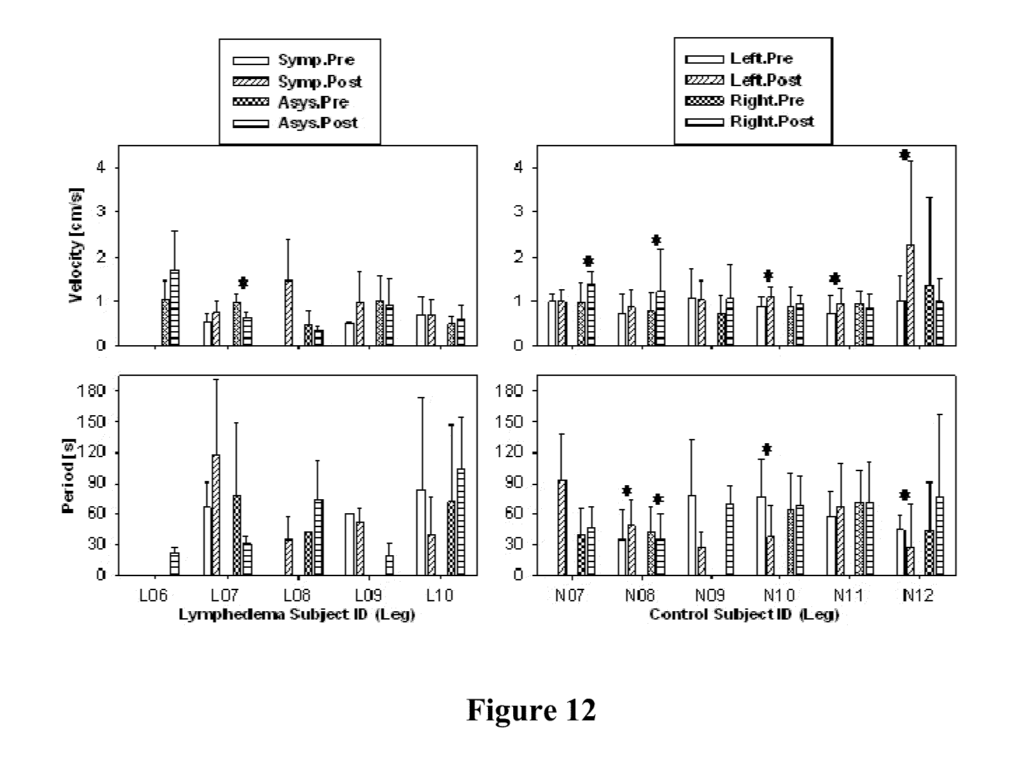

The pre- and post-MLD average of apparent lymph velocities and propulsion periods obtained from the symptomatic and asymptomatic legs of 5 LE subjects and the control legs of 6 normal subjects are shown in FIG. 12. None of the symptomatic legs showed a statistically significant change in apparent lymph velocity after MLD, and one asymptomatic leg (subject L07) showed a significant decrease in lymph velocity. None of the legs of LE subjects showed significant changes in propulsion period. However, three LE subjects (L07, L09, and L10) experienced improved function via either increase in apparent lymph velocity and/or decrease in propulsions period in symptomatic leg. LE subject L06 was not clinically responsive to MLD, and no lymph propulsion was found in the symptomatic leg, but a vessel, which was not seen before MLD, appeared near the buttock in the image after MLD. Apparently, the imaging agent was pushed into this “newly” visible vessel by MLD, but still no propulsion was observed. The apparent lymph velocities exhibited statistically significant increase after MLD in five of the 12 control legs, and the periods showed statistically significant decrease after MLD in four. It is noteworthy that there were a total of 28 and 40 pulses observed in five symptomatic legs, pre- and post-MLD; 47 and 56 pulses in five asymptomatic legs, pre- and post-MLD; and 297 and 469 pulses in 12 control legs, pre- and post-MLD.

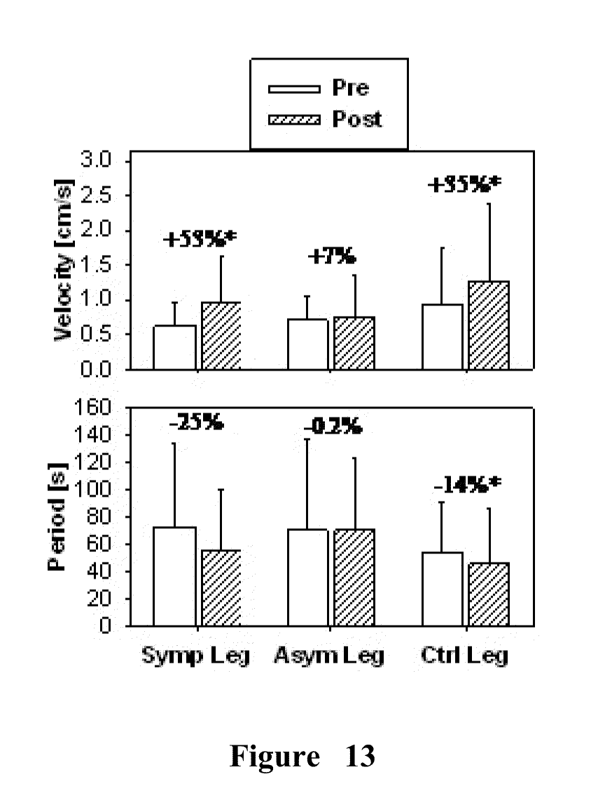

The statistical results of the pooled data grouped into symptomatic, asymptomatic, and control legs are shown in FIG. 13. Average apparent lymph velocities and propulsion periods are plotted for each group, and the percentages of changes from pre- to post-MLD are indicated. The apparent lymph velocities increased for all groups after MLD, and the increases were statistically significant in symptomatic and control legs (Table 6). The propulsion periods for the control leg group showed a statistically significant decrease after MLD.

For the statistical analysis results of the pooled data from all 22 subjects (44 limbs) in the study, ANOVA indicates that MLD significantly improved the lymphatic function as reported by increases in apparent velocities in all limb diagnoses (control +28% p<0.001, symptomatic +23% p=0.003, and asymptomatic +25% p<0.001) and by reduction in the propulsion period in the limbs of normal subjects (−23% p<0.001).

### Example 3

### The Use of NIR Fluorescence Imaging to Direct Manual Decongestive Therapy in a Cancer Survivor with Impaired Lymphatic Function

An African-American male subject 54 years of age was enrolled under a single patient IND to test the ability to identify functional lymphatic drainage pathways following injections of NIR fluorescent dye in his face and neck. His medical history included chemoradiation therapy for a T3 squamous cell carcinoma of the soft palate in March, 2005. He subsequently developed radiation fibrosis of the neck, complicated by severe facial edema. In April, 2008, he developed recurrent disease in the left tongue and right mandible; he subsequently underwent a right hemimandibulectomy, left partial glossectomy, and left floor of the mouth resection with construction of a free flap in June of 2008. He underwent subsequent debridement of the surgical site. He experienced significant pain in the facial area, and he was diagnosed with lymphedema in December 2008; he underwent surgical debulking of the free flap later that month. Therapy was attempted with a facial compression mask, but the subject was unable to wear it due to pain. Manual decongestive therapy has been attempted with some decrease in swelling, but without knowing if there are functional lymphatics present and if there are, where they are located, the benefit of the therapy is questionable. The subject has been able to open his mouth slightly, but he is unable to open it wide enough to eat, given the swelling that includes his tongue.

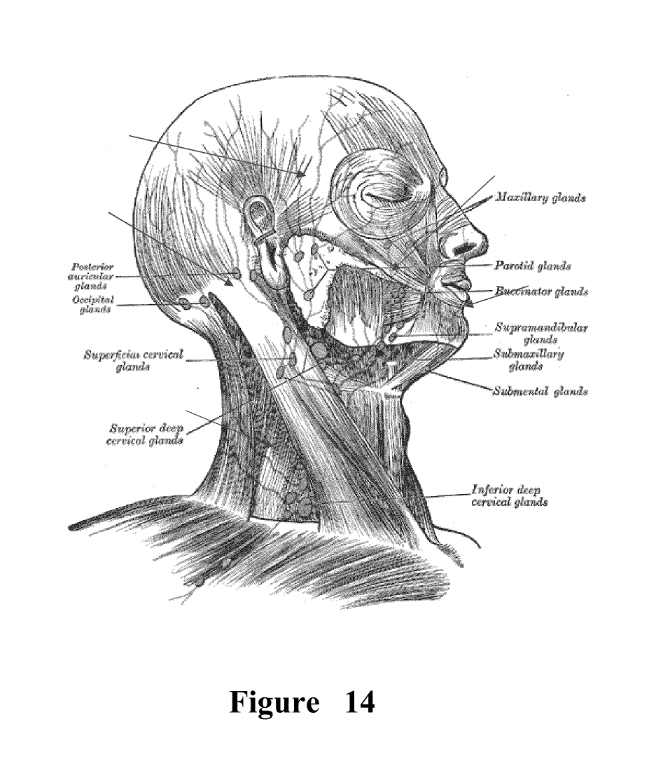

Nine injections of ICG (25 mcg/injection) were made in the face and neck in two stages. Initially, the subject received 4 injections of ICG, with 5 subsequent injections approximately 1.5 h later (total dose 225 mcg). The subject placed protective glasses on that blocked all light to the eyes, and fluorescence imaging was initiated after the first 4 injections using a 785 nm laser diode light incident upon the tissue in the region of injection. Mounted on a steerable, articulating arm, the expanded beam was scanned over the site of injection and along the neck. The excitation light activated ICG resulting in tissue fluorescence that propagated to the tissue surface. The fluorescent light was collected onto the photocathode of an ICCD system also mounted on the articulating arm and outfitted with an interference filter to collect the ICG fluorescence via a lens that was focused upon the tissue surface. Imaging was conducted prior to massage therapy, during massage therapy, and after massage had been completed. The ICG injection sites are indicated in FIG. 14. With the exception of the injection in the lower lip, corresponding injections were performed on the other side of the head and neck.

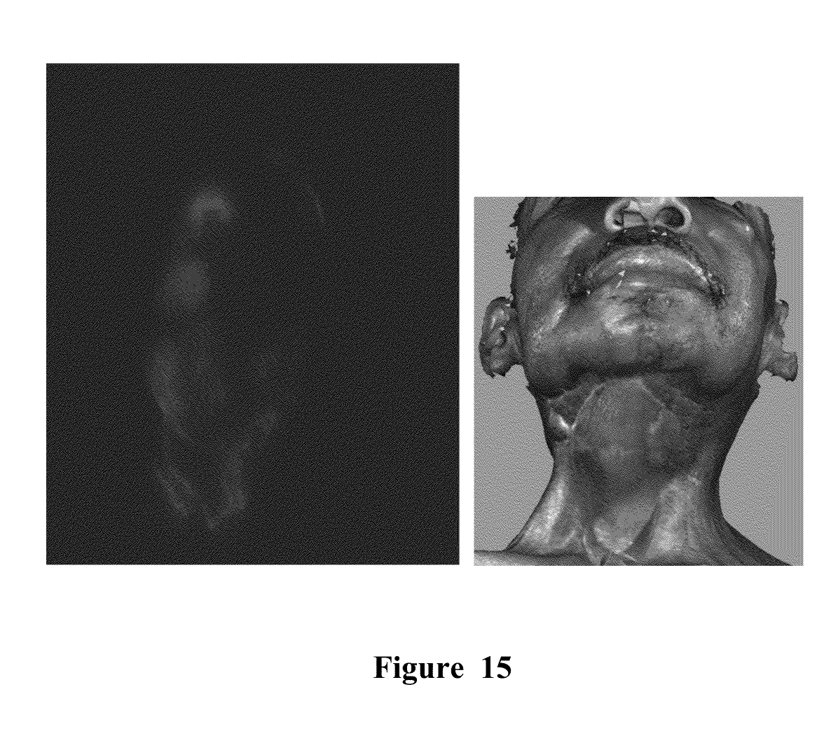

Lymph channels in the front of his neck were observed shortly after the first four injections were made. The channels were in defined areas, and they tended to avoid some surgical scars, although there were instances of transport of ICG through the lymph across scars on the neck. An example of the images obtained during the procedure is shown in FIG. 15. Multiple images were obtained with different orientations to visualize lymph flow throughout the head and neck region of the subject and provided information for how massage should be directed to push fluid into the functioning lymphatic vessels.

Lymphatic channels were found on the back and front of the neck, but there was surprisingly no flow to the axillary lymph node basin was observed nor were any fluorescent lymph nodes in the axillary evident in the preliminary review of data. The imaging suggests that functional draining lymph channels did exist on the front and back of the neck. These channels could provide a route for direction of manual lymphatic drainage through massage.

After imaging, manual massage was performed with imaging of the area being massaged and after massage had been completed. Preliminary results show that propulsive flow did occur, especially on the left side of the face.

### Example 4

### The Use of NIR Fluorescence Imaging to Assess and Monitor Abnormal Function in a Lymphatic Disorder

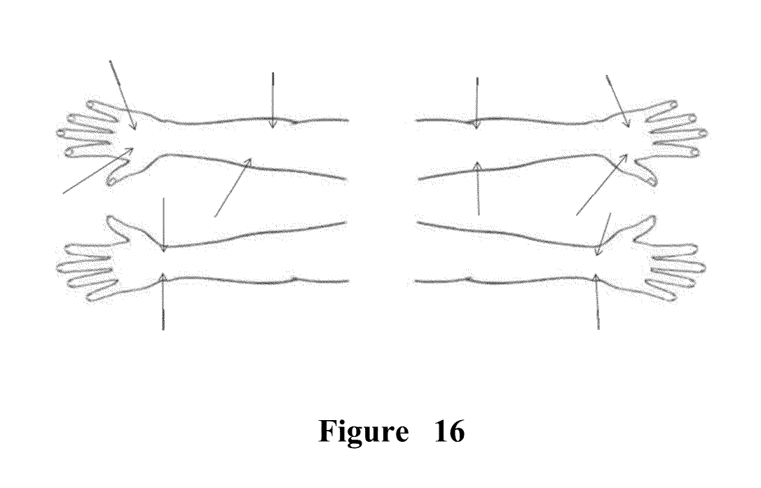

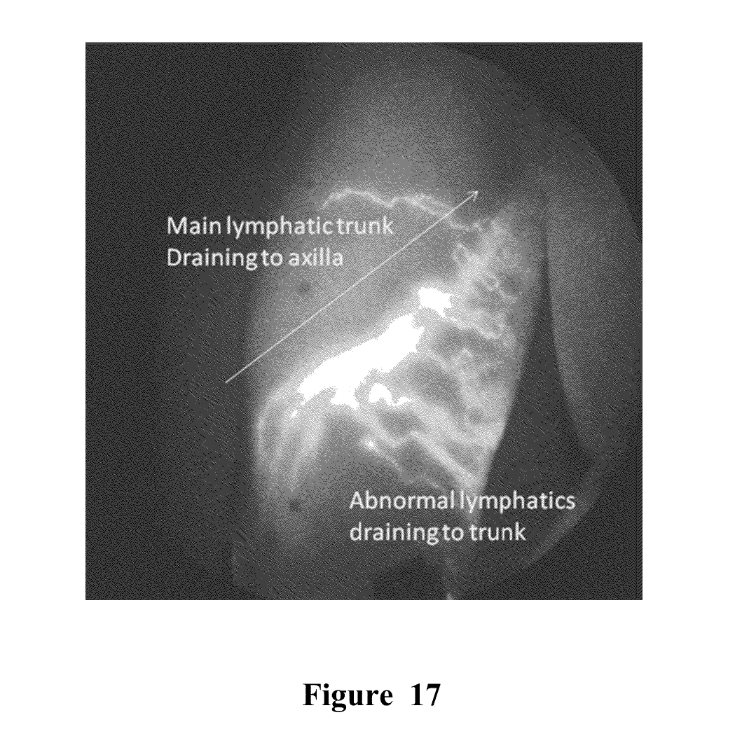

NIR fluorescence imaging A 67 year of age male with a history of carpel tunnel surgery in 2008, congestive heart failure (CHF) in 2006, Hodgkin's disease in 1966, and Radiation treatment in 1967 presented with right upper extremity lymphedema one month before NIR fluorescence imaging. The sights of injection of NIR fluorophore into both afflicted and un-afflicted arms as indicated in FIG. 16. On his afflicted side, NIR fluorescence imaging revealed lymphatics functioning normally, exhibiting a pulsing action which propelled lymphatic fluid to his axilla. In addition, a presumably new abnormal lymphatic vessel formation that drained into the main lymphatic trunk was also observed (FIG. 17). NIR fluorescence imaging of his asymptomatic side visualized normal propulsive behavior in most of his lymphatics, but also visualized retrograde or backward flow not typically seen in normal subjects (see FIG. 17). Lymphatic reflux and back flow may be akin to venous reflux, as currently assessed with Doppler ultrasound to evaluate venous insufficiency. These results suggest that the localized symptoms of lymphedema seen on his right arm may be as result of a systemic lymphatic defect as evidenced by abnormal lymphatic function in his left hand. This indicates that NIR lymphatic imaging may be used to assess lymphovascular insufficiency as well as to assess the relative contribution of the lymphatics in vascular disorders.

### Example 5

### The Use of NIR Fluorescence Imaging to Identify and Predict Patients at Risk for Developing a Lymphatic Disorder as a Result of Surgery or Therapies

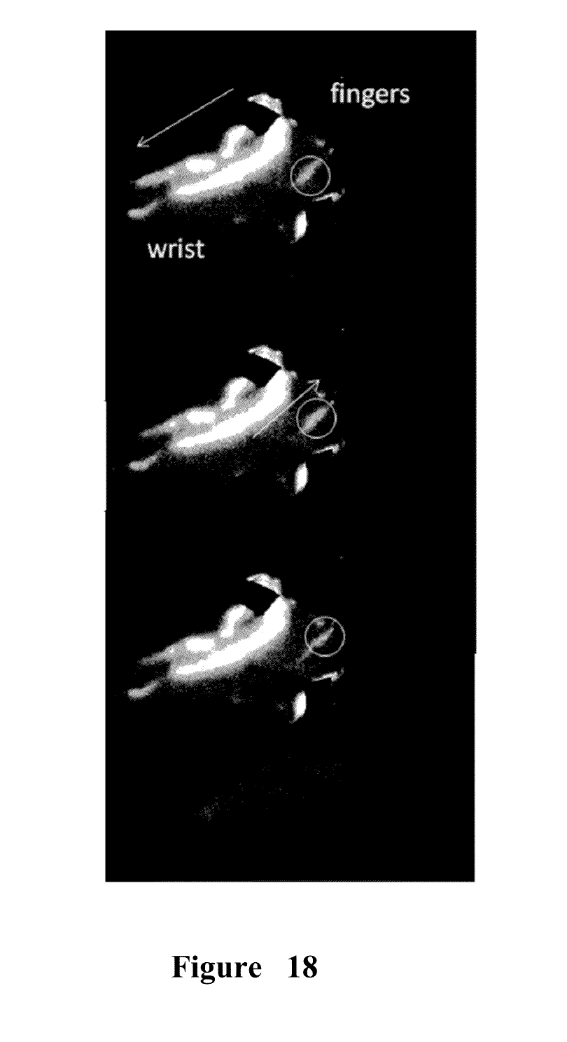

The subject described above in Example 4, presented with upper right extremity lymphedema that was accompanied by abnormal vessel architecture in the afflicted arm which was the limb in which carpel tunnel surgery occurred in 2008, see for example FIG. 18, which shows NIR fluorescence imaging of the asymptomatic arm of the subject suffering from early lymphedema and retrograde flow). Also, as described above in Example 1, patients with unilateral lymphedema were found to have compromised lymphatic function not only in their symptomatic limb, but also in the asymptomatic limb when compared to normal control subjects. Without being bound by theory or any particular mechanism, it is thought that this may indicate that there is a systemic predisposition for the development of lymphedema, lymphatic disorders and lymphovascular disease. Clearly the development and progression of such lymphatic disorders can be assessed using the NIR fluorescence functional lymph imaging as disclosed herein. Thus, NIR fluorescence imaging of lymphoid function may be used as a method to pre-screen those cancer patients who may develop lymphatic disorders, such as lymphedema, as a result of diagnostic or therapeutic nodal staging. Patients determined to be at risk of developing lymphatic disorders, may wish to decline elective surgery if they are at increased risk of lymphedema. In some cases such patients may be prescribed to undergo prophylactic treatment based upon the risk identified through NIR fluorescence lymphatic imaging.

### Example 6

### Integrin α9β1 as a Target for Molecular Imaging of Lymphangiogenesis

Lymphangiogenesis is the process by which lymph vessels form in response to disease, injury, or cancer metastasis. The process of new blood vessel formation (called angiogenesis) had been previously imaged by the targeting contrast agents to the integrin αᵥβ₃ (“αᵥβ₃”) expressed on proliferating blood endothelial cells (BECs). In the present studies it was discovered that integrin α₉β₁ (or “α₉β₁”) is also expressed on proliferating lymph endothelial cells (LECs) during adult lymphangiogenesis and that it can be targeted with an imaging conjugate to image the process of lymphangiogenesis with MR fluorescence and/or radioactivity. A method is presented to non-invasively visualize newly formed lymph vessels utilizing α₉β₁ expression in lymph vessels as a marker of lymphangiogenesis, and utilizing new imaging agents (e.g., conjugates) to bind α₉β₁ as a way to detect lymphangiogenesis.

While there are several ligands for α₉β₁, the extracellular matrix protein tenascin C sequence Pro-Leu-Ala-Glu-Ile-Asp-Gly (PLAEIDG) (SEQ ID NO: 1), which is known to bind to α₉β₁, was chosen for use in some embodiments. This peptide is described by H. Schneider et al. FEBS Letters, Volume 429, Issue 3, Pages 269-273, 1998, and incorporated herein by reference. The peptide was labeled with a near-infrared fluorophore, IRDye® 800CW (LI-COR BIOSCIENCES, Lincoln, Nebr.) that excites at 780 nm and emits at 830 nm and used it to detect lymphangiogenesis in vivo as well as activation of lymphatic endothelial cells in vitro. The IRDye® 800CW dye bears an NHS ester reactive group that will couple to proteins and form a stable conjugate. Fluorescent conjugates labeled with IRDye 800CW display an absorption maximum of 774 nm and an emission maximum of 789 nm in 1×PBS. These spectral characteristics match the 800 nm channel on the Odyssey and Aerius as well as the sensitive clinical imaging systems described and used in the studies disclosed herein in addition to IRDye® 800CW, those of skill in the art would readily recognize that alternative contrast dyes might be used similarly to the described embodiments. Such contrast agents include compounds, such as organic dyes that exhibit fluorescence at near-infrared wavelengths when exposed to excitation light. Examples of such contrast agents which may be used in conjunction with the one or more binding moieties include without limitation, α₉β₁ include, but are not limited to, tricarbocyanine dyes, bis(carbocyanine)dyes, dicarbocyanine dyes, indol-containing dyes, polymethine dyes, acridines, anthraquinones, benzimidazols, indolenines, napthalimides, oxazines, oxonols, polyenes, porphyrins, squaraines, styryls, thiazols, xanthins as well as other NIR contrast agents known to those of skill in the art, such as IR-783, or combinations thereof. In various embodiments, one or more such contrast agents may be used in conjunction with the one or more binding moieties to other pertinent molecules associated with lymphatic function and lymphangiogenesis other than α₉β₁ integrin. Herein the use of IRDye800-GGGPLAEIDGIELTY (SEQ ID NO: 2) comprising a peptide known to bind α₉ρ₁, (H. Schneider et al. 1998, ibid) for imaging lymphangiogenesis as well as activation of LECs is shown as a representative example. These IRDye800-integrin binding peptide conjugates are referred to herein as IRDye800-GGGPLAEIDGIELTY (SEQ ID NO: 2).”

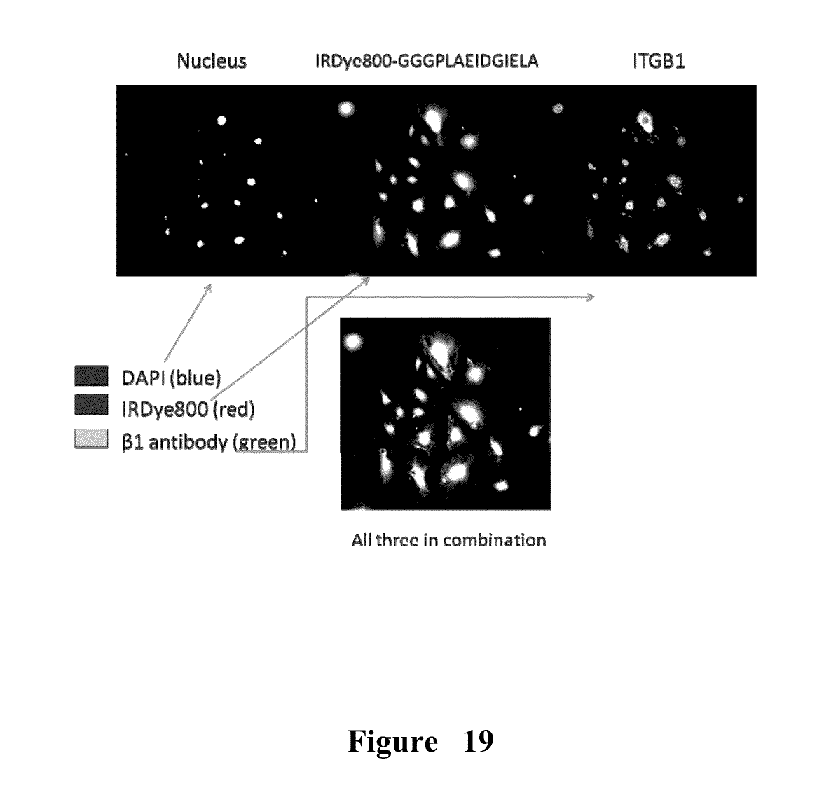

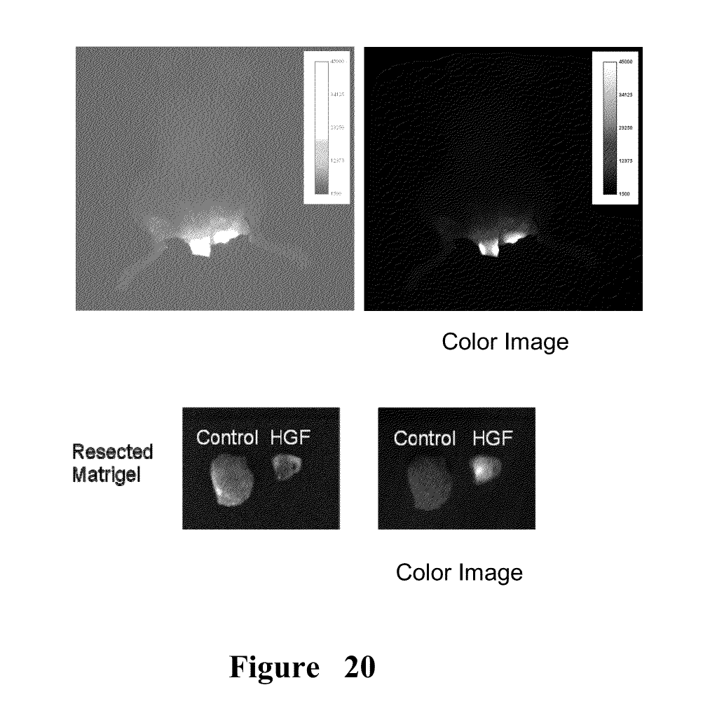

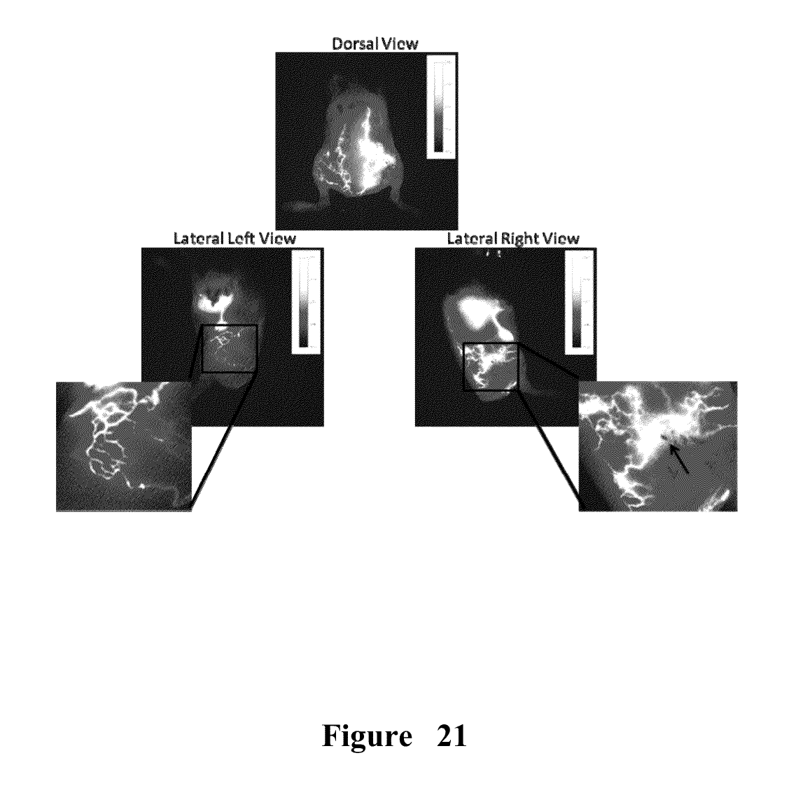

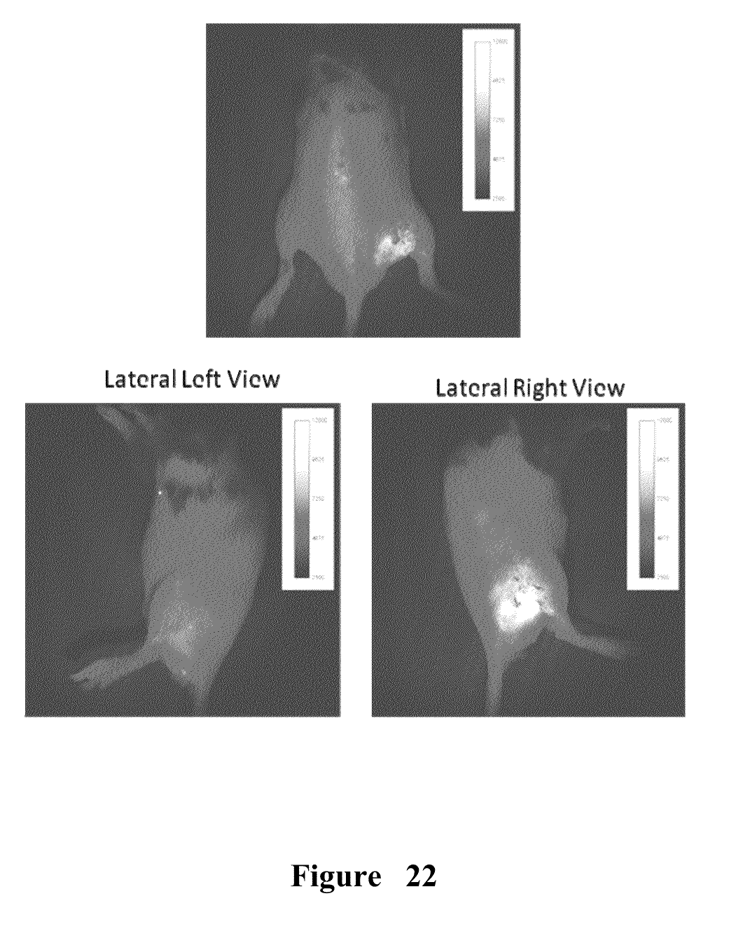

This targeting approach can be used to detect the process of lymphangiogenesis in animal models in, for example, drug discovery programs focusing on cessation of tumor lymphangiogenesis (such is associated with cancer metastasis), or as a diagnostic to detect the lymphangiogenesis that occurs in metastatic cancer and other lymphovascular disorders. Lymphangiogenesis and the accompanying activation of LECS are known to be stimulated by vascular endothelial growth factor-C (VEGF-C) or hepatic growth factor (HGF). FIG. 19 shows the binding of IRDye800-GGGPLAEIDGIELTY (SEQ ID NO: 2) to LECs incubated in VEGF-C for 30 minutes after which the cells were stained with a nuclear stain, IRDye800-IBP, and an antibody to the β1 subunit on α₉β₁. The image for the combined stains showed that IRDye800-IBP co-localized with the antibody to 131, indicating specificity of binging to α₉β₁ and upregulation of α₉β₁ upon LEC stimulation by VEGF-C. When VEGF-C is not present during incubation, α₉β₁ (over) expression and IRDye800-GGGPLAEIDGIELTY (SEQ ID NO: 2) binding are not seen. Similar results are occur with HGF stimulation of LECs. FIG. 20 shows a dorsal view of the fluorescence in a mouse model of lymphangiogenesis following intradermal administration of the image conjugate (IRDye800-GGGPLAEIDGIELTY (SEQ ID NO: 2)) at the base of the tail. Implanted on the base of the right hind limb (seen in the lower portion of the figure) is a MATRIGEL plug with HGF, known to induce lymphangiogenesis and on the left hind limb, the same MATRIGEL plug without HGF. The induced lymphangiogenesis is clearly visualized by the increase in fluorescence 24 hours through NIR imaging after the administration of imaging conjugate, IRDye800-GGGPLAEIDGIELTY (SEQ ID NO: 2) binding to the animal. Upon resecting the MATRIGEL plugs, the MATRIGEL plug with the HGF showed fluorescence due to IRDye800-GGGPLAEIDGIELTY (SEQ ID NO: 2) indicative of LEC activation and lymphangiogenesis, while the control matrigel plug with no HGF present did not show appreciable fluorescence due to IRDye800-GGGPLAEIDGIELTY (SEQ ID NO: 2). In yet another animal model of lymphangiogenesis caused by inflammation, preferential binding of IRDye800-GGGPLAEIDGIELTY (SEQ ID NO: 2) as well as new lymph architecture is shown following induction of skin inflammation. FIG. 21 shows the lymphatic imaging conducted with intradermal administration of ICG (as shown above in human studies) on a mouse. The ht side of the animal had skin inflammation induced by topical application of Oxazolone, which induces skin inflammation while the left side had none. The dorsal view (top) shows a greater number of lymphatic vessels on the right side that is associated with Oxazolone-induced inflammation and lymphangiogenesis. Increased vessel density, leakiness, and dilated vessels on the inflamed side as compared to the untreated side of the animal as evident from the lymphatic imaging. In the same animal, IRDye800-GGGPLAEIDGIELTY (SEQ ID NO: 2) was injected intravenously and 24 hours later, NIR fluorescence imaging was conducted to assess increased update associated with those tissues that were the site of Oxazolone application and increased lymphatic vessel densities that are indicative of lymphangiogenesis. FIG. 22 shows the dorsal view showing increased fluorescence associated with IRDye800-GGGPLAEIDGIELTY (SEQ ID NO: 2) in the right region associated with skin inflammation. These results combined suggest that imaging agents targeting α₉β₁ can be used to non-invasively image lymphangiogenesis that occurs in inflammation, injury, as well as potentially cancer metastasis.

The new imaging agents for detecting lymphangiogenesis can be used, for example, in preclinical studies to evaluate therapeutics intended to halt the lymphangiogenesis that accompanies cancer metastases as well as to evaluate therapeutics that encourage or block the process of lymphangiogenesis for use in the treatment of lymphangiogenesis that accompany, but are not limited to, injury or surgery, or lymphedema and lymphedema-related disorders. This imaging conjugate and NIR fluorescence may be used for the diagnosis of human disorders and metastatic cancer and without the need to perform lymph node biopsy. The imaging conjugate may be modified to enhanced stability as well as to add additional agents for hybrid medical imaging, including MR, CT, PET, SPECT, or gamma scintigraphy.

In some embodiments, a new imaging agent targeted to lymph endothelial cell integrin α₉β₁ is provided. An exemplary imaging agent is a conjugate comprising a peptide derived from the extracellular matrix protein tenascin C sequence, which is known to bind to the lymph endothelial cell integrin α₉β₁. The peptide is labeled with a near-infrared fluorophore, and the resulting conjugate is used to detect lymphangiogenesis in vivo as well as activation of lymphatic endothelial cells in vitro. Lymph endothelial cell integrin α₉β₁ expression may be related to the beginnings of tumor formation. As such, embodiments of the imaging agents may be used to stain lymph structures for detailed imaging of lymph architecture as well as serving as potential markers for tumor angiogenesis, tumor metastases, etc. (see for example Kwon and Sevick-Muraca, 2010, Functional lymphatic imaging in tumor-bearing mice. J Immunol Methods. 2010 August 31; 360(1-2):167-72. Epub June 30).

### Example 7

### NW Imaging Agent Development

Clinical, near-infrared (NIR) fluorescence imaging of human lymphatics using the tricarbocynanine dye, indocyanine green (ICG) and comparatively compact MR imaging instrumentation is clearly demonstrated in this disclosure as well as in the scientific literature (see for example, Lymphatic imaging in humans with near-infrared fluorescence. Rasmussen, et al, Curr Opin Biotechnol. February; 20 (1):74-82. Epub 2009 Feb. 23 2009 and Marshall et al. 2010 (“Near-infrared fluorescence imaging in humans with indocyanine green: a review and update,” Open Surgical Oncology Journal, in press)). MR fluorescence with excitation at >750 nm is advantageous for deep tissue imaging owing to high signal to noise ratio, minimal light absorption, maximal tissue penetration, and low background due to minimal autofluorescence. ICG has maximal absorption at 780 nm and is approved for human use in clinical vascular and hepatic function testing on the basis of its dark green color and its ability to associate with albumin. Upon properly designing instrumentation for sensitive collection of its fluorescence, human lymphatic imaging with rapid image acquisition (200 milliseconds) under conditions of ICG micro dosing after intradermal injections of as little as 25 ug have been done. However, due to its instability in solution and low quantum efficiency ICG can be a poor fluorophore and the fluorescent signals from deep lymphatics such as the saphenous lymphatic channel in the lower limb can be very dim. As a result, alternative probe systems such as liposomes and nanoparticles coupled to red-excitable dyes such as Cy5.5 have been pursued. However Cy5.5, with an excitation wavelength of 690 and emission at 710 nm, also has limited tissue penetration. Therefore, in order to develop a brighter, but low MW NIR fluorescent conjugate that was capable of deep tissue imaging while associating with serum proteins for retention within vascular spaces, an albumin binding domain (ABD) peptide (RLIEDICLPRWGCLWEDD (SEQ ID NO:3)) was employed. This albumin binding domain peptide is described by Dennis et al. 2002 in “Albumin binding as a general strategy for improving the pharamacokinetics of proteins.” J Biol Chem. 2002 Sep. 20; 277(38):35035-43, which is hereby incorporated herein by reference. Below, are described the optical properties of NIR fluorophores and their application in pre-clinical murine lymphatic imaging. In vitro studies confirmed the higher extinction coefficient and quantum efficiency of IRDye800 agents as compared to commonly used ICG, and in vivo studies indicate that IRDye800 conjugates are taken up and retained in the lymphatics and can be used to provide high sensitivity imaging.

Agent Preparation:

ICG was obtained from Akorn (Lake Forest, Ill.) and reconstituted in sterile phosphate buffered saline (PBS) immediately prior to use. IRDye800CW NHS ester was purchased and IRDye800-MSA (mouse serum albumin) (with a dye to protein ratio of 2.8) was provided in kind from LI-COR Biosciences (Lincoln, Nebr.). DyLight800 was obtained from Thermo Scientific (Rockford, Ill.). agents were stored in lyophilized forms at −20° C. upon arrival. Immediately before use, each was reconstituted in sterile PBS to desired concentration. The ABD peptide, RLIEDICLPRWGCLWEDD (SEQ ID NO:3), >95% purity, was obtained from New England Peptide (Gardner, Mass.), used as received, and reconstituted immediately before use in Phosphate Buffered. Saline (pH 7.4). ABD peptide conjugation to IRDye800CW was performed by adding a 1.75 times molar excess of IRDye800CW to ABD peptide. Conjugation occurred over 1 hour at 37 degrees on a rotator platform under dark conditions. The conjugated peptide was then purified and analyzed using a Zorbax 80SB-C18 HPLC column in TEAA buffer and stored at 4° C. and protected from light.

Characterization of Imaging Agent:

Excitation and emission spectra (Horiba Jobin Yvon, Edison, N.J.) of 1 μM solutions of ICG, IRDye800CW, IRDye800-MSA, IRDye800-ABD and DyLight800 were obtained at wavelengths of 785 and 830 nm respectively, with an integration time of 0.3 seconds Extinction coefficients were determined from the slope of absorbance at 785 nm as a function of serial dilutions of each agent. Fluorescence quantum yield was determined by the comparative method of Williams et al. 1983 (Williams, Winfield and Miller. Analyst 109, 1067, 1983) using the quantum yield of ICG (in water) at 785/830 nm (24) as a standard. Lifetime measurements were obtained using the frequency domain-method using ICG as a standard. The IC50 of IRDye800-ABD was determined using a competitive binding assay modified from Dennis et al. 2002 (ibid). Briefly, MAXISORP fluorescent plates (Nunc, Rochester, N.Y.) were coated with mouse serum albumin (Sigma St Louis, Mo.) and incubated overnight at 4 degrees. Plates were washed three times with PBS+0.05% Tween20 (Sigma St. Louis, Mo.) to remove unbound albumin and were then blocked with TBS in casein (Pierce Rockford, Ill.) for 1 hour at 25 degrees. Equal volumes of unlabeled and labeled peptide were combined in centrifuge tubes for five different concentrations (0.05-10 nM) of unlabeled ABD peptide. The resulting peptide solution was then plated on the albumin coated plate, covered, and incubated for 1-2 hours at 37 degrees. The plate was washed with PBS+0.05% Tween20 three times to remove unbound peptide and fluorescence was measured using the ODYSSEY fluorescent plate reader (LI-COR, Lincoln, Nebr.). The average and standard deviation of four IC-50 values were determined.

Characterization of Imaging Agents:

Table 7 lists the MW, excitation/emission maxima, extinction coefficient at 785 nm, quantum yield at 785/830 (ex/em), and lifetime for ICG, IRDye800CW, IRDye800-MSA, and IRDye800-ABD, and DyLight 800, at 785/830 (ex/em). In addition, Table 1 lists the relative brightness of each compound relative to ICG which was computed by taking the ratio of ((extinction coefficient)×(quantum yield))/((extinction coefficient of ICG)×(quantum yield of ICG)). The results show that IRDye800 itself is approximately 22 times brighter than ICG and that on a molar basis the IRDye800 conjugates retain their brightness over ICG. In addition, the lifetime of IRDye800 conjugates remains similar to one another, indicating IRDye800 is a consistent fluorophore when conjugated onto proteins (i.e., mouse serum albumin) or peptides (ABD). It was not determined that the extinction coefficient or quantum yield of 1 ICG solution increased with the addition of albumin, although it is well known that at higher solution concentrations ICG self-quenches and the addition of albumin or aspartic acid can improve fluorescent yield of ICG. The DyLight 800 was also 10.7 times brighter than ICG, but less bright than IRDye800. As a consequence, DyLight800 was not further characterized nor conjugated to ABD. Spectra for ICG and the IRDye800 conjugates were determined.

Affinity for Serum Proteins:

After conjugation with IRDye800 and purification, to determine if the ABD peptide conjugate retained its affinity for albumin by comparing its IC-50 for mouse serum albumin to that reported by Dennis et al. 2002 (Albumin binding as a general strategy for improving the pharamacokinetics of proteins. J Biol Chem. 2002 Sep. 20; 277(38):35035-43). The ABD peptide was previously found to bind albumin with high affinity at a site distinct from albumin's other important ligand binding sites, thus avoiding competitive inhibition of albumin's essential molecular interactions with other compounds. This peptide was found to enhance vascular retention of several proteins and may also have the ability to enhance the lymphatic retention of IRDye800 for lymphatic imaging. IRDye800-ABD conjugate to had an IC-50 of 1.19+1-0.89 nM which compared favorably to the IC-50 of unconjugated ABD peptide of 5+/−2 nM as reported by Dennis et al., 2002 (ibid). Thus, ABD-IRDye800 conjugate retained its high affinity for albumin binding.

In vivo studies: All animal studies were approved by the University of Texas Health Science Center—Houston, Center for Laboratory Animal Medicine and Care (CLAMC) animal welfare committee. Mice were housed in the university's AAALAC-I accredited facilities under sterile conditions according to CLAMC policy. Animal studies were conducted using shaved 6-7 week old female, C57BL/6 mice (Harlan, Charles River). Mice were divided into experimental groups, anesthetized with isofluorane inhalation, and injected with ICG, IRDye800, IRDye800-MSA or IRDye800-ABD. Each mouse was injected intradermally at the base of the tail with 20 μL of 500 uM ICG or 20 μL of 100 μM dye for IRDye800, IRDy800-MSA and IRDye800-ABD in order to assess their performance in vivo. The injections were all made by the same investigator and the site was covered with sterile tape immediately prior to imaging to prevent camera saturation.

The white light in vivo imaging revealed the injection site at the base of the tail and a “roadmap” indicative of normal lymphatics in the C57BL/6 mice. Animals were illuminated by a laser diode (100 mA, 80 mW for 785 nm, Sanyo; Richmond, Ind.) with the light source expanded to a circular area approximately 8 cm in diameter. Fluorescence emission was collected using an Electron Multiplying Charge Coupled Device (EMCCD) camera with a holographic notch-plus band-rejection filter (785-nm center wavelength for ICG) and a bandpass filter (830-nm center wavelength for ICG) placed prior to the lens to reject back-scattered and reflected excitation light. Fluorescence images were acquired with an integration time of 200 ms. For registration purposes, “white light” images were taken by removing the filters and acquiring images over 500 ms integration time. Up to 750 fluorescent images and 3 white light images were acquired immediately after injection on the left and right side of each animal. Statistical analyses of differences in fluorescent intensity were assessed by student t-test in which α=0.05. To quantify lymphatic clearance of each agent, the fluorescence intensity within the region containing the inguinal lymph node was recorded at 0.5 min, 2.5 min, 15 min, 30 min and 50 min after injection of each NIR agent. Images were processed using V++ software (Digital Optics; Auckland, New Zealand), and data was analyzed to determine in vivo fluorescent intensities in select regions of interest (ROI) using IMAGEJ (NIH, Bethesda, Md.) and MATLAB (The Mathworks). Statistical analysis was conducted using the student t-test with α=0.05.

In vivo lymphatic imaging studies utilizing an escalating dose of IC was done. It was determined that at 20 and 100 uM concentrations of ICG, the lymphatic vessels and nodes were dim and difficult to discern. In contrast, at 500 μM, the lymphatic vasculature became brighter and more prominent. A plexus of minor lymphatic vessels became visible in the lower trunk region and the inguinal lymph node, as well as the collecting lymphatic vessels leading up toward the axillary lymph node were easily identified. Thus, it was determined that the 500 μM concentration of ICG most clearly depicted lymphatic architecture in vivo and would be used for further studies. Dose escalation imaging was also conducted with IRDye800 and it was found that 100 μM IRDye800, a five-fold lower concentration of dye than needed for ICG was sufficient to delineate lymphatic structures.

Lymphatic Clearance Study:

In an additional study, he lymphatic clearance rate of ICG, IRDye800, IRDye800-MSA and IRDye800-ABD from the inguinal lymph node was determined. Initially, IRDye800 had a significantly higher intensity in the inguinal node than IRDye800-MSA. By 30 minutes however, IRDye800 had a significantly lower fluorescent intensity than both IRDye800-MSA and IRDye800-ABD. Conversely, both IRDye800-ABD and IRDye800-MSA started out with a lower intensity suggesting a slower filling of the node, but ended with a significantly higher intensity, showing slowed clearance. ICG seemed to follow a similar trend as IRDye800-MSA but the difference in intensity compared to IRDye800 did not reach significance. The more rapid clearance of IRDye800 may be attributed to the fact that unlike ICG and IR800-MSA; IRDye800 exists as a free dye in vivo and does not associate with albumin or similar proteins that may aid in lymphatic retention (Ohnishi, et al, 2005, Organic Alternatives to quantum dots for intraoperative near-infrared fluorescent sentinel lymph node mapping. Molecular Imaging 2005 July; 4:3 172-181). As the selected NIR dye is intended to be used clinically for lymphatic imaging, it is important that it is able to remain in the lymphatics for an extended period of time to allow for patient imaging. Thus based on these initial results, IRDye800-MSA and IRDye800-ABD are the most favorable imaging agents for additional studies. However, the large size of IRDye800-MSA (or IRDye800-HSA), as well as potential issues with chemistry, manufacturing and control of a large protein, may limit its clinical applicability. Therefore, IRDye800-ABD exhibits favorable optical properties, lymphatic uptake, and retention appears to be a good fluorophore for lymphatic imaging.

Without further elaboration, it is believed that one skilled in the art can, using the description herein, utilize the present invention to its fullest extent. The embodiments described herein are to be construed as illustrative and not as constraining the remainder of the disclosure in any way whatsoever. While the preferred embodiments have been shown and described, many variations and modifications thereof can be made by one skilled in the art without departing from the spirit and teachings of the invention. For example, although the described embodiments illustrate use of the present compositions and methods on humans, those of skill in the art would readily recognize that these methods and compositions could also be applied to veterinary medicine and other mammals. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

TABLE 1
Demographic Characteristics of Subjects Imaged

| | Normal | | |
|---|---|---|---|
| Characteristic | (n = 24) | Lymphedema (n = 20) | |
| Gender, n (%) | | | |
| Male | 5 (20.8) | 1 (5.0) | |
| Female | 19 (79.2) | 19 (95.0) | |
| Mean age, years ± SD* | 38.2 ± 11.0 | 49.7 ± 17.6 | |
| Mean weight, kg ± SD* | 72.7 ± 12.1 | 74.3 ± 21.3 | |
| Body surface area†, | 1.8 ± 0.2 | 1.8 ± 0.2 | |
| m² ± SD* | | | |
| Ethnicity, n (%) | | | |
| Caucasian | 19 (79.2) | 15 (75.0) | |
| Black | 2 (8.3) | 3 (15.0) | |
| Asian | 2 (8.3) | 1 (5.0) | |
| Hispanic | 1 (4.2) | 1 (5.0) | |
| Other | 0 (0.0) | 0 (0.0) | |
| Limb, n (%) | | | |
| Leg | 12 (50.0) | 10 (50.0) | |
| Arm | 12 (50.0) | 10 (50.0) | |
| Etiology of disease, | | | |
| n (%) | | Arms (n = 10) | Legs (n = 10) |
| Primary | — | 0 (0.0) | 3 (30.0) |
| Post-surgical | — | 10 (100.0) | 3 (30.0) |
| Post-injury | — | 0 (0.0) | 2 (20.0) |
| Insect bite | — | 0 (0.0) | 2 (20.0) |

*SD indicates standard deviation,
†Body surface area calculated by the DuBois formula

TABLE 2
Summary of Subject Demographics

| Subject | | | | Dose ICG | | | |
|---|---|---|---|---|---|---|---|
| ID | Age | Gender | Ethnicity* | (μg) | Limb | Diagnosis | Comments^{†} |
| C01 | 46 | F | C | 400 | Leg | Control | |
| C02 | 46 | F | C | 375 | Arm | Control | |
| C03 | 23 | M | C | 262.5 | Leg | Control | |
| C04 | 59 | M | C | 375 | Arm | Control | |
| C05 | 29 | F | C | 400 | Leg | Control | |
| C06 | 22 | M | C | 200 | Arm | Control | |
| C07 | 27 | F | C | 350 | Leg | Control | |
| C08 | 41 | F | C | 200 | Arm | Control | |
| C09 | 29 | F | C | 375 | Leg | Control | |
| C10 | 46 | F | C | 200 | Arm | Control | |
| C11 | 35 | F | C | 400 | Leg | Control | |
| C12 | 50 | F | AA | 200 | Arm | Control | |
| C13 | 52 | F | C | 350 | Leg | Control | |
| C14 | 36 | F | C | 200 | Arm | Control | |
| C15 | 40 | F | A | 400 | Leg | Control | |
| C16 | 48 | F | C | 200 | Arm | Control | |
| C17 | 35 | F | AA | 200 | Arm | Control | |
| C18 | 25 | F | C | 400 | Leg | Control | |
| C19 | 34 | F | C | 400 | Leg | Control | |
| C20 | 41 | F | H | 300 | Arm | Control | |
| C21 | 22 | M | C | 400 | Leg | Control | |
| C22 | 35 | F | C | 300 | Arm | Control | |
| C23 | 59 | F | C | 400 | Leg | Control | |
| C24 | 37 | M | A | 300 | Arm | Control | |
| L01 | 18 | F | C | 400 | Leg | Lymphedema | |
| L02 | 46 | F | C | 400 | Arm | Lymphedema | |
| L03 | 66 | F | C | 300 | Arm | Lymphedema | |
| L04 | 66 | F | C | 400 | Leg | Lymphedema | |
| L05 | 62 | F | C | 300 | Arm | Lymphedema | |
| L06 | 36 | F | C | 400 | Leg | Lymphedema | |
| L07 | 20 | F | C | 400 | Leg | Lymphedema | |
| L08 | 48 | F | C | 312.5 | Arm | Lymphedema | |
| L09 | 56 | F | H | 325 | Arm | Lymphedema | |
| L10 | 23 | F | AA | 400 | Leg | Lymphedema | |
| L11 | 68 | F | C | 300 | Arm | Lymphedema | |
| L12 | 48 | F | AA | 350 | Leg | Lymphedema | |
| L13 | 40 | F | AA | 400 | Leg | Lymphedema | |
| L14 | 67 | F | C | 300 | Arm | Lymphedema | |
| L15 | 31 | M | C | 400 | Leg | Lymphedema | |
| L16 | 47 | F | C | 50 | Leg | Lymphedema | Subject declined additional |
| | | | | | | | injections. |
| L17 | 65 | F | A | 400 | Leg | Lymphedema | |
| L18 | 81 | F | C | 250 | Arm | Lymphedema | |
| L19 | 57 | F | C | 300 | Arm | Lymphedema | |
| L20 | 49 | F | C | 300 | Arm | Lymphedema | |

*A = Asian, AA = African American, C = Caucasian, and H = Hispanic
^{†}Comments are only included when something remarkable is noted.

TABLE 3
Summary of Velocity And Period In Control Subjects

| | | | | Velocity (cm/s) | | | | | Period (s) | | | | | | |
|---|---|---|---|---|---|---|---|---|---|---|---|---|---|---|---|
| | | | | | | | Mean (Upper, | | Number | | | | p-value^{†} | | |
| Subject ID | Side | Limb | Diagnosis* | No. of Pulses | Range | Median | Lower Deviation) | p-value^{†} (corrected) | of Periods | Range | Median | Mean | (corrected) | Features^{‡} | Comments§ |
| C01 | Left | Leg | Cont | 44 | 0.3-2.7 | 0.8 | 0.9 | | 20 | 15-93 | 30.5 | 33.7 (20.8, | 0.013 | T | Subject |
| | | | | | | | (0.5, 1.5) | | | | | 54.6) | (0.34) | | reported |
| | Right | Leg | Cont | 36 | 0.3-1.6 | 0.9 | 0.8 | | 22 | 7-167 | 69.0 | 56.3 (26.1, | | | previous |
| | | | | | | | (0.6, 1.2) | | | | | 121.4) | | | chemical |
| | | | | | | | | | | | | | | | burn in |
| | | | | | | | | | | | | | | | area of |
| | | | | | | | | | | | | | | | tortuosity. |
| C02 | Left | Arm | Cont | 23 | 0.4-2.7 | 0.7 | 0.8 | | 12 | 9-66 | 36.5 | 34.0 (18.8, | | | |
| | | | | | | | (0.5, 1.3) | | | | | 61.6) | | | |
| | Right | Arm | Cont | 19 | 0.3-1.0 | 0.8 | 0.7 | | 14 | 15-65 | 20.0 | 22.6 (15.9, | | | |
| | | | | | | | (0.5, 0.9) | | | | | 32.1) | | | |
| C03 | Left | Leg | Cont | 27 | 0.6-6.1 | 1.9 | 1.9 | 0.014 | 16 | 8-154 | 13.6 | 22.8 (7.8, | | | |
| | | | | | | | (1.1, 3.3) | (0.45) | | | | 66.7) | | | |
| | Right | Leg | Cont | 8 | 0.8-1.8 | 1.3 | 1.3 | | 0 | | | | | | |
| | | | | | | | (0.9, 1.8) | | | | | | | | |
| C04 | Left | Arm | Cont | 18 | 0.3-2.6 | 0.7 | 0.7 | | 9 | 12.6-62.9 | 17.8 | 20.6 (12.5, | | | |
| | | | | | | | (0.4, 1.3) | | | | | 33.9) | | | |
| | Right | Arm | Cont | 42 | 0.3-2.6 | 0.7 | 1.3 | | 23 | 11.0-39.7 | 23.9 | 21.4 (14.1, | | | |
| | | | | | | | (0.9, 1.8) | | | | | 32.3) | | | |
| C05 | Left | Leg | Cont | 45 | 0.3-1.4 | 0.6 | 0.6 | | 10 | 31.4-118.2 | 45.6 | 57.1 (35.6, | | | |
| | | | | | | | (0.4, 0.9) | | | | | 91.5) | | | |
| | Right | Leg | Cont | 42 | 0.3-1.9 | 0.7 | 0.7 | | 15 | 14.2-95.1 | 46.3 | 42.8 (24.0, | | | |
| | | | | | | | (0.5, 1.0) | | | | | 76.0) | | | |
| C06 | Left | Arm | Cont | 135 | 0.3-2.6 | 0.9 | 0.9 | | 57 | 3.4-117.1 | 42.2 | 39.9 (19.5, | 0.022 | | |
| | | | | | | | (0.6, 1.2) | | | | | 81.6) | (0.58) | | |
| | Right | Arm | Cont | 105 | 0.4-4.9 | 0.9 | 0.9 | | 31 | 9.5-81.4 | 30.3 | 28.3 (15.1, | | | |
| | | | | | | | (0.6, 1.3) | | | | | 53.0) | | | |
| C07 | Left | Leg | Cont | 68 | 0.4-3.2 | 0.9 | 1.0 | | 16 | 9.6-169.0 | 63.1 | 58.2 (28.6, | | | |
| | | | | | | | (0.6, 1.5) | | | | | 118.3) | | | |
| | Left, | Leg | Cont | 1 | −1.5 | −1.5 | −1.5 | | 0 | | | | | R | |
| | Back | | | | | | | | | | | | | | |
| | Right | Leg | Cont | 15 | 0.3-1.6 | 0.8 | 0.7 | | 1 | 78.3 | 78.3 | 78.3 | | | |
| | | | | | | | (0.4, 1.2) | | | | | | | | |
| C08 | Left | Arm | Cont | 36 | 0.4-2.5 | 1.0 | 1.1 | 0.0040 | 11 | 15.0-82.6 | 56.4 | 42.1 (22.9, | | | |
| | | | | | | | (0.7, 1.6) | (0.14) | | | | 77.2) | | | |
| | Right | Arm | Cont | 87 | 0.3-3.1 | 0.8 | 0.8 | | 32 | 8.9-138.2 | 25.7 | 29.7 (13.3, | | | |
| | | | | | | | (0.6, 1.2) | | | | | 66.4) | | | |
| C09 | Left | Leg | Cont | 29 | 0.5-2.0 | 0.8 | 0.8 | | 6 | 41.2-110.6 | 56.6 | 60.6 (40.3, | | | Tortuous |
| | | | | | | | (0.6, 1.2) | | | | | 91.1) | | | vessel in |
| | Left, | Leg | Cont | 1 | −0.4 | −0.4 | −0.4 | | 0 | | | | | R | ankle |
| | Back | | | | | | | | | | | | | | which |
| | Right | Leg | Cont | 23 | 0.5-1.4 | 0.7 | 0.7 | | 5 | 15.5-97.5 | 57.5 | 43.7 (21.6, | | T | subject had |
| | | | | | | | (0.5, 1.1) | | | | | 88.4) | | | previously |
| | | | | | | | | | | | | | | | sprained. |
| C10 | Left | Arm | Cont | 32 | 0.4-2.1 | 0.8 | 0.8 | | 13 | 3.5-199.6 | 47.0 | 39.2 (13.2, | | | |
| | | | | | | | (0.6, 1.2) | | | | | 116.2) | | | |
| | Right | Arm | Cont | 46 | 0.4-1.8 | 0.8 | 0.8 | | 11 | 10.5-43.1 | 29.1 | 26.6 (16.3, | | | |
| | | | | | | | (0.6, 1.2) | | | | | 43.4) | | | |
| C11 | Left | Leg | Cont | 5 | 0.8-1.2 | 0.9 | 1.0 | | 0 | | | | | | |
| | | | | | | | (0.8, 1.2) | | | | | | | | |
| | Right | Leg | Cont | 19 | 0.3-2.1 | 0.8 | 0.9 | | 7 | 15.9-85.3 | 28.5 | 33.6 (18.1, | | | |
| | | | | | | | (0.5, 1.4) | | | | | 62.5) | | | |
| C12 | Left | Arm | Cont | 47 | 0.4-1.3 | 0.8 | 0.8 | | 34 | 18.5-157.7 | 28.6 | 29.9 (20.8, | 0.011 | T | |
| | | | | | | | (0.6, 1.0) | | | | | 43.1) | (0.32) | | |
| | Right | Arm | Cont | 105 | 0.3-1.1 | 0.8 | 0.8 | | 75 | 7.7-106.1 | 22.8 | 24.2 (15.4, | | | |
| | | | | | | | (0.6, 1.0) | | | | | 38.0) | | | |
| C13 | Left | Leg | Cont | 53 | 0.2-2.4 | 0.7 | 0.7 | | 17 | 10.3-96.6 | 20.9 | 25.3 (12.1, | | T | |
| | | | | | | | (0.4, 1.2) | | | | | 52.6) | | | |
| | Right | Leg | Cont | 45 | 0.2-1.2 | 0.6 | 0.6 (0.4, | | 22 | 10.1-132.3 | 35.9 | 33.7 (18.3, | | EF | |
| | | | | | | | 0.9) | | | | | 62.1) | | | |
| | Right, | Leg | Cont | 1 | −0.4 | −0.4 | −0.4 | | 0 | | | | | R | |
| | Back | | | | | | | | | | | | | | |
| C14 | Left | Arm | Cont | 112 | 0.2-4.3 | 0.9 | 0.8 | | 53 | 2.3-85.0 | 29.5 | 26.6 (12.9, | 0.0013 | | |
| | | | | | | | (0.5, 1.3) | | | | | 54.7) | (0.039) | | |
| | Right | Arm | Cont | 36 | 0.3-2.3 | 0.8 | 0.8 | | 18 | 12.5-116.7 | 74.6 | 54.5 (25.8, | | | |
| | | | | | | | (0.5, 1.2) | | | | | 114.9) | | | |
| C15 | Left | Leg | Cont | 35 | 0.2-2.3 | 0.6 | 0.7 | | 15 | 6.9-125.0 | 33.2 | 28.2 (14.4, | | | |
| | | | | | | | (0.4, 1.0) | | | | | 55.3) | | | |
| | Right | Leg | Cont | 45 | 0.3-1.9 | 0.6 | 0.7 | | 29 | 13.8-110.2 | 33.9 | 36.4 (21.2, | | | |
| | | | | | | | (0.4, 1.1) | | | | | 62.7) | | | |
| C16 | Left | Arm | Cont | 37 | 0.3-1.6 | 0.7 | 0.7 | | 10 | 7.7-133.0 | 42.2 | 38.5 (13.8, | | | |
| | | | | | | | (0.5, 1.0) | | | | | 107.1) | | | |
| | Right | Arm | Cont | 75 | 0.4-1.7 | 0.8 | 0.8 | | 42 | 9.3-173.4 | 40.1 | 37.7 (18.7, | | | |
| | | | | | | | (0.6, 1.0) | | | | | 75.8) | | | |
| C17 | Left | Arm | Cont | 12 | 0.2-0.7 | 0.4 | 0.4 | | 3 | 41.0-54.8 | 45.2 | 46.7 (40.2, | | | |
| | | | | | | | (0.3, 0.6) | | | | | 54.1) | | | |
| | Right | Arm | Cont | 15 | 0.2-0.8 | 0.5 | 0.5 | | 6 | 30.5-57.4 | 39.8 | 41.2 (31.3, | | | |
| | | | | | | | (0.4, 0.7) | | | | | 54.2) | | | |
| C18 | Left | Leg | Cont | 42 | 0.2-2.5 | 0.8 | 0.9 | | 21 | 3.2-193.8 | 82.4 | 53.4 (18.6, | | | |
| | | | | | | | (0.5, 1.7) | | | | | 153.7) | | | |
| | Right | Leg | Cont | 3 | 0.3-1.1 | 0.7 | 0.6 | | 0 | | | | | | |
| | | | | | | | (0.3, 1.2) | | | | | | | | |
| C19 | Left | Leg | Cont | 20 | 0.4-1.4 | 0.9 | 0.8 | | 12 | 12.3-125.4 | 76.9 | 65.2 (32.8, | | | |
| | | | | | | | (0.6, 1.1) | | | | | 129.6) | | | |
| | Right | Leg | Cont | 17 | 0.3-1.8 | 0.8 | 0.8 | | 5 | 32.5-110.3 | 51.3 | 57.0 (32.9, | | | |
| | | | | | | | (0.4, 1.3) | | | | | 98.7) | | | |
| C20 | Left | Arm | Cont | 61 | 0.2-2.4 | 0.7 | 0.7 | | 33 | 13.1-77.9 | 30.3 | 30.8 (20.6, | | | |
| | | | | | | | (0.5, 1.1) | | | | | 46.1) | | | |
| | Right | Arm | Cont | 48 | 0.1-1.5 | 0.9 | 0.8 | | 28 | 15.8-71.5 | 26.4 | 29.5 (20.5, | | | |
| | | | | | | | (0.5, 1.3) | | | | | 42.6) | | | |
| C21 | Left | Leg | Cont | 30 | 0.1-1.7 | 0.8 | 0.6 | 0.037 | 10 | 27.6-107.4 | 58.0 | 54.3 (35.8, | | T, H | Small |
| | | | | | | | (0.3, 1.2) | (0.99) | | | | 82.3) | | | abnormalities |
| | Right | Leg | Cont | 12 | 0.4-1.3 | 1.0 | 0.9 | | 3 | 50.0-107.0 | 55.4 | 66.7 (44.1, | | | near one |
| | | | | | | | (0.6, 1.3) | | | | | 100.8) | | | injection |
| | | | | | | | | | | | | | | | site on calf. |
| C22 | Left | Arm | Cont | 21 | 0.3-1.1 | 0.5 | 0.6 | | 15 | 27.0-208.9 | 55.4 | 54.3 (30.3, | | | |
| | | | | | | | (0.4, 0.8) | | | | | 97.4) | | | |
| | Right | Arm | Cont | 131 | 0.2-1.0 | 0.6 | 0.6 | | 107 | 16.1-188.7 | 36.7 | 40.8 (25.2, | | | |
| | | | | | | | (0.5, 0.7) | | | | | 66.2) | | | |
| C23 | Left | Leg | Cont | 36 | 0.4-2.7 | 0.9 | 0.9 | | 12 | 28.3-83.3 | 40.5 | 42.3 (30.8, | | | |
| | | | | | | | (0.5, 1.4) | | | | | 58.0) | | | |
| | Right | Leg | Cont | 33 | 0.1-11.7 | 1.0 | 0.9 | | 9 | 4.8-152.6 | 31.9 | 25.8 (7.9, | | | |
| | | | | | | | (0.3, 2.4) | | | | | 83.5) | | | |
| C24 | Left | Arm | Cont | 75 | 0.4-1.0 | 0.6 | 0.6 | 0.0011 | 51 | 7.0-205.1 | 71.3 | 62.7 (30.5, | | | |
| | | | | | | | (0.5, 0.7) | (0.039) | | | | 128.8) | | | |
| | Right | Arm | Cont | 175 | 0.1-1.2 | 0.5 | 0.5 | | 115 | 9.0-250.5 | 77.7 | 68.5 (35.9, | | | |
| | | | | | | | (0.4, 0.7) | | | | | 130.6) | | | |

*Cont = Control
^{†}p values provided for significant differences between left and right limbs. The values in parenthesis have been corrected for multiple comparisons using the Holm test.
^{‡}Features noted include tortuous vessels (T), hyperplastic lymphatic networks (H), extravascular fluorescence (EF), and lymphatic reflux (R).
^{§}Comments are only included when something remarkable is noted.

TABLE 4
Summary of Velocity and Period In Subjects With Lymphedema

| | | | | | | | | | Period (s) | | | | | | |
|---|---|---|---|---|---|---|---|---|---|---|---|---|---|---|---|
| | | | | Velocity (cm/s) | | | | | Number of | | | | p-value^{†} | | |
| Subject ID | Side | Limb | Diagnosis* | Number of Pulses | Range | Median | Mean | p-value^{†} (corrected) | Periods | Range | Median | Mean | (corrected) | Features^{‡} | Comments |
| L01 | Left | Leg | Symp | 0 | | | 0 | NC | 0 | | | | | EF | Onset at age 18 |
| | Right | Leg | Asym | 3 | 0.6-1.5 | 1.0 | 1.0 | | 0 | | | | | | with unknown |
| | | | | | | | (0.6, | | | | | | | | etiology (no record |
| | | | | | | | 1.5) | | | | | | | | of trauma or |
| | | | | | | | | | | | | | | | surgical procedure). |
| L02 | Left | Arm | Symp | 0 | | | 0 | 1.9e−4§ | 0 | | | | | EF | Onset after |
| | Left, | Arm | Symp | 12 | −4.2-−0.5 | −1.0 | −1.5 | | 9 | 15.9-24.3 | 20.9 | 20.7 | | ABP | mastectomy. Had |
| | Back | | | | | | (−2.8, −0.2) | | | | | (18.0, | | | spontaneous fistula |
| | | | | | | | | | | | | 23.4) | | | for lymph drainage |
| | Right | Arm | Asym | 5 | 0.2-1.0 | 0.5 | 0.5 | | 0 | | | | | | in palm of |
| | | | | | | | (0.3, | | | | | | | | symptomatic hand. |
| | | | | | | | 0.8) | | | | | | | | |
| L03 | Left | Arm | Asym | 50 | 0.5-3.3 | 0.9 | 0.9 | 1.8e−7 | 27 | 3.5-78.8 | 31.5 | 30.8 | | EF | Onset after |
| | | | | | | | (0.6, | (7.1e−6) | | | | (14.5, | | | mastectomy. |
| | | | | | | | 1.4) | | | | | 65.4) | | | |
| | Right | Arm | Symp | 2 | 0.2-0.2 | 0.2 | 0.2 | | 1 | 56.9 | 56.9 | 56.9 | | EF, H, T | |
| | | | | | | | (0.2, | | | | | | | | |
| | | | | | | | 0.2) | | | | | | | | |
| L04 | Left | Leg | Symp | 11 | 0.2-0.8 | 0.6 | 0.5 | 0.0015 | 6 | 43.8-113.0 | 60.5 | 62.5 | | H, T | Onset after cancer |
| | | | | | | | (0.3, | (0.055) | | | | (44.6, | | | surgery. |
| | | | | | | | 0.8) | | | | | 87.5) | | | |
| | Right | Leg | Asym | 15 | 0.6-1.3 | 1.0 | 0.9 | | 7 | 25.6-200.5 | 45.9 | 57.0 | | | |
| | | | | | | | (0.8, | | | | | (25.2, | | | |
| | | | | | | | 1.2) | | | | | 128.8) | | | |
| L05 | Left | Arm | Asym | 10 | 0.5-1.6 | 0.9 | 0.8(0.5, | 0.018 | 7 | 11.3-117.0 | 37.6 | 33.4 | | H, R | Onset after |
| | | | | | | | 1.3) | (0.55) | | | | (13.0, | | | mastectomy (Right), |
| | | | | | | | | | | | | 86.2) | | | had second |
| | Left, | Arm | Asym | 2 | −1.2-−0.6 | −0.9 | −0.9 | | 1 | 67.4-67.4 | | 67.4 | | H, R | mastectomy (Left) 9 |
| | Back | | | | | | (−1.3, −0.5) | | | | | | | | years after first. |
| | Right | Arm | Symp | 10 | 0.4-4.1 | 1.6 | 1.6 | | 9 | 8.4-54.6 | 12.0 | 16.1 | | EF, H | Edema commenced |
| | | | | | | | (0.8, | | | | | (9.0, | | | ~1 year prior to |
| | | | | | | | 3.0) | | | | | 28.8) | | | second |
| | | | | | | | | | | | | | | | mastectomy. |
| L06 | Left | Leg | Symp | 14 | 0.5-0.8 | 0.7 | 0.6 | | 10 | 24.4-135.3 | 64.0 | 54.1 | | H, T, | Onset after trauma |
| | | | | | | | (0.6, | | | | | (31.1, | | EF | (foot sprain). |
| | | | | | | | 0.8) | | | | | 94.2) | | | |
| | Right | Leg | Asym | 35 | 0.3-1.5 | 0.7 | 0.7 | | 24 | 20.7-117.4 | 48.1 | 43.5 | | | |
| | | | | | | | (0.4, | | | | | (25.3, | | | |
| | | | | | | | 1.0) | | | | | 74.6) | | | |
| L07 | Left | Leg | Symp | 62 | 0.3-1.9 | 0.8 | 0.8 | | 32 | 18.6-216.4 | 58.2 | 62.2 | | H, T, | Onset after multiple |
| | | | | | | | (0.5, | | | | | (33.5, | | EF | leg injuries. |
| | | | | | | | 1.3) | | | | | 115.3) | | | |
| | Right | Leg | Asym | 49 | 0.2-2.7 | 1.0 | 1.0 | | 19 | 13.2-133.2 | 40.2 | 44.0 | | | |
| | | | | | | | (0.5, | | | | | (21.8, | | | |
| | | | | | | | 1.8) | | | | | 88.8) | | | |
| L08 | Left | Arm | Symp | 13 | 0.6-2.2 | 1.1 | 1.0 | 0.045 | 8 | 11.0-82.5 | 44.4 | 38.7 | 2.0e−4 | EF | Onset after |
| | | | | | | | (0.7, | (1.0) | | | | (21.7, | (6.1e−3) | | melanoma surgery. |
| | | | | | | | 1.5) | | | | | 68.9) | | | Bilateral |
| | Right | Arm | Asym | 19 | 0.5-1.3 | 0.8 | 0.8 | | 6 | 6.4-15.3 | 10.7 | 10.4 | | | lymphedema on |
| | | | | | | | (0.6, | | | | | (7.5, | | | legs. |
| | | | | | | | 1.0) | | | | | 14.5) | | | |
| L09 | Left | Arm | Asym | 57 | 0.2-1.2 | 0.5 | 0.6 | 0.0045 | 33 | 16.5-117.3 | 38.1 | 38.0 | 1.5e−5 | | Onset after |
| | | | | | | | (0.4, | (0.15) | | | | (20.7, | (4.8e−4) | | mastectomy. |
| | | | | | | | 0.9) | | | | | 69.6) | | | |
| | Right | Arm | Symp | 14 | 0.2-1.4 | 0.4 | 0.4 | | 11 | 18.7-24.5 | 22.2 | 22.1(20.4, | | H, T | |
| | | | | | | | (0.2, | | | | | 23.9) | | | |
| | | | | | | | 0.6) | | | | | | | | |
| L10 | Left | Leg | Symp | 0 | | | 0 | NC | 0 | | | | | EF | Onset occurred |
| | Right | Leg | Asym | 6 | 0.2-1.1 | 0.4 | 0.4 | | 2 | 41.7-42.5 | 42.1 | 42.1 | | T, H | after bug bite. |
| | | | | | | | (0.2, | | | | | (41.5, | | | |
| | | | | | | | 0.8) | | | | | 42.7) | | | |
| L11 | Left | Arm | Symp | 18 | 0.4-3.1 | 0.8 | 0.8 | 0.019 | 10 | 14.5-75.0 | 32.5 | 32.0 | | EF, H, T | Onset after |
| | | | | | | | (0.5, | (0.56) | | | | (17.7, | | | mastectomy. |
| | | | | | | | 1.5) | | | | | 57.7) | | | |
| | Right | Arm | Asym | 38 | 0.3-1.6 | 0.6 | 0.6 | | 14 | 11.9-140.0 | 35.1 | 35.6 | | | |
| | | | | | | | (0.4, | | | | | (18.0, | | | |
| | | | | | | | 0.8) | | | | | 70.5) | | | |
| L12 | Left | Leg | Symp | 2 | 0.5-0.5 | 0.5 | 0.5 | | 1 | 60.4 | 60.4 | 60.4 | | EF, H, T | Unknown etiology, |
| | | | | | | | (0.5, | | | | | | | | possibly due to |
| | | | | | | | 0.5) | | | | | | | | pressure from large, |
| | Right | Leg | Asym | 3 | 0.6-1.6 | 0.7 | 0.9 | | 0 | | | | | H, T | untreated fibroid |
| | | | | | | | (0.5, | | | | | | | | cyst in uterine |
| | | | | | | | 1.5) | | | | | | | | system. |
| L13 | Left | Leg | Asym | 20 | 0.1-0.8 | 0.5 | 0.5 | | 8 | 12.4-229.5 | 36.7 | 44.8 | | T | Onset after spider |
| | | | | | | | (0.3, | | | | | (15.6, | | | bite on toe, also had |
| | | | | | | | 0.7) | | | | | 128.5) | | | fibroid cysts in |
| | Right | Leg | Symp | 15 | 0.1-1.4 | 0.7 | 0.6 | | 6 | 6.0-228.3 | 41.2 | 37.5 | | H, T, | groin. |
| | | | | | | | (0.3, | | | | | (7.9, | | EF | |
| | | | | | | | 1.2) | | | | | 178.8) | | | |
| L14 | Left | Arm | Symp | 1 | 0.8 | 0.8 | 0.8 | | 0 | | | | | EF, H, | Onset after |
| | Right | Arm | Asym | 192 | 0.1-4.9 | 0.9 | 0.8 | | 99 | 2.8-200.0 | 27.8 | 25.3 | | | lumpectomy and |
| | | | | | | | (0.5, | | | | | (11.7, | | | axillary dissection. |
| | | | | | | | 1.4) | | | | | 54.7) | | | |
| L15 | Left | Leg | Symp | 11 | 0.4-1.9 | 1.3 | 1.1 | 0.0064 | 7 | 18.6-102.3 | 83.9 | 67.9 | | H, T | Onset at age 10 |
| | | | | | | | (0.7, | (0.21) | | | | (37.7, | | | with unknown |
| | | | | | | | 1.9) | | | | | 122.1) | | | etiology. |
| | Right | Leg | Asym | 12 | 0.5-1.0 | 0.6 | 0.7 | | 6 | 31.2-109.5 | 46.3 | 54.7 | | T | |
| | | | | | | | (0.5, | | | | | (31.3, | | | |
| | | | | | | | 0.8) | | | | | 95.9) | | | |
| L16 | Left | Leg | Symp | 7 | 0.3-0.8 | 0.5 | 0.5 | | 6 | 27.5-141.0 | 65.1 | 62.6 (35.7, | | EF, H | Onset after multiple |
| | | | | | | | (0.3, | | | | | 109.7) | | | surgeries in pelvic |
| | | | | | | | 0.6) | | | | | | | | region. |
| | Right | Leg | Asym | 6 | 0.2-0.8 | 0.3 | 0.3 | | 0 | | | | | | |
| | | | | | | | (0.2, | | | | | | | | |
| | | | | | | | 0.5) | | | | | | | | |
| L17 | Left | Leg | Asym | 45 | 0.1-2.3 | 0.7 | 0.7 | 0.011 | 25 | 31.3-252.0 | 77.7 | 74.6 | | H, T | Onset after multiple |
| | | | | | | | (0.3, | (0.36) | | | | (45.3, | | | surgeries in pelvic |
| | | | | | | | 1.4) | | | | | 123.0) | | | region. |
| | Right | Leg | Symp | 11 | 0.2-0.9 | 0.4 | 0.4 | | 4 | 28.3-146.3 | 47.9 | 55.5 (26.5, | | H, T | |
| | | | | | | | (0.3, | | | | | 116.3) | | | |
| | | | | | | | 0.7) | | | | | | | | |
| L18 | Left | Arm | Asym | 49 | 0.2-1.2 | 0.6 | 0.5 | 3.6e−7 | 24 | 22.3-111.0 | 49.7 | 48.8 (31.1, | 1.40e−8 | | Onset after |
| | | | | | | | (0.4, | (1.4e−5) | | | | 76.5) | (4.5e−7) | | mastectomy. |
| | | | | | | | 0.8) | | | | | | | | |
| | Right | Arm | Symp | 10 | 0.7-1.2 | 0.9 | 0.9 | | 9 | 16.1-24.7 | 23.5 | 21.7 | | H, T | |
| | | | | | | | (0.7, | | | | | (18.4, | | | |
| | | | | | | | 1.1) | | | | | 25.5) | | | |
| L19 | Left | Arm | Asym | 54 | 0.4-2.9 | 0.8 | 0.9 | 0.023 | 19 | 5.7-139.4 | 56.9 | 42.6 | | | Onset after |
| | | | | | | | (0.6, | (0.64) | | | | (17.2, | | | mastectomy. |
| | | | | | | | 1.2) | | | | | 105.7) | | | |
| | Right | Arm | Symp | 12 | 0.1-1.7 | 0.5 | 0.4 | | 1 | 25.0 | 25.0 | 25.0 | | EF, H, T | |
| | | | | | | | (0.2, | | | | | | | | |
| | | | | | | | 1.1) | | | | | | | | |
| | Right | Arm | Symp | 1 | −0.3 | | −0.3 | | 0 | | | | | EF, H, T | |
| | Back | | | | | | | | | | | | | | |
| L20 | Left | Arm | Symp | 45 | 0.5-2.0 | 0.8 | 0.8 | 4.3e−4 | 39 | 5.0-84.0 | 36.1 | 33.1 | | EF, H, T | Onset after |
| | | | | | | | (0.6, | (0.016) | | | | (17.7, | | | mastectomy. |
| | | | | | | | 1.1) | | | | | 61.8) | | | |
| | Right | Arm | Asym | 255 | 0.1-2.5 | 0.6 | 0.6 | | 172 | 2.8-148.4 | 27.7 | 29.9 | | | |
| | | | | | | | (0.4, | | | | | (14.3, | | | |
| | | | | | | | 1.0) | | | | | 62.5) | | | |

*Asym = asymptomatic, Symp = Symptomatic, and Cont = Control
^{†}p values provided for significant differences between asymptomatic and symptomatic limbs, NC indicates a non-calculated significant difference due to a zero velocity. The values in parenthesis have been corrected for multiple comparisons using the Holm test.
^{‡}Features noted include tortuous vessels (T), hyperplastic lymphatic networks (H), extravascular fluorescence (EF), lymphatic reflux (R), and active backward propulsion (ABR).
§p value from t-test of non-transformed forward flow in asymptomatic limb and active backward flow in symptomatic limb.

TABLE 5
Summary of Subject Demographics

| | | | | | | | | Pre-MLD | | | | Post-MLD | | | |
|---|---|---|---|---|---|---|---|---|---|---|---|---|---|---|---|
| | | | | | | | | Number | | Number of | | Number | | Number of | |
| Subject | | | | Dose | | | | of pulses | Range of | periods between | Range of | of pulses | Range of | periods between | Range of |
| ID | Age | Gender | Ethnicity* | (μg ICG) | Limb | Diagnosis | Side | (velocity) | velocity (cm/s) | pulses | period (s) | (velocity) | velocity (cm/s) | pulses | period (s) |
| L01 | 46 | F | C | 400 | Arm | LE: onset after | Symp | 0 | N/A | 0 | N/A | 0 | N/A | 0 | N/A |
| | | | | | | mastectomy | Asys | 5 | 0.2-1.0 | 0 | N/A | 88 | 0.4-2.8 | 31 | 4-95 |
| L02 | 62 | F | C | 300 | Arm | LE: onset after | Symp | 10 | 0.4-4.1 | 9 | 8-55 | 0 | N/A | 0 | N/A |
| | | | | | | mastectomy (right), | Asys | 10 | 0.5-1.6 | 7 | 11-117 | 16 | 0.4-1.8 | 10 | 5-54 |
| | | | | | | had 2nd | | | | | | | | | |
| | | | | | | mastectomy (left) 9 | | | | | | | | | |
| | | | | | | years after 1st. | | | | | | | | | |
| L03 | 48 | F | C | 312.5 | Arm | LE: onset after | Symp | 13 | 0.6-2.2 | 8 | 11-83 | 53 | 0.4-2.8 | 32 | 6-127 |
| | | | | | | melanoma surgery | Asys | 19 | 0.5-1.3 | 6 | 6-15 | 104 | 0.3-1.5 | 78 | 8-100 |
| L04 | 56 | F | H | 325 | Arm | LE: onset after | Symp | 14 | 0.2-1.4 | 11 | 19-25 | 5 | 0.2-0.5 | 4 | 36-46 |
| | | | | | | mastectomy | Asys | 57 | 0.2-1.2 | 33 | 17-117 | 18 | 0.3-1.0 | 10 | 31-81 |
| L05 | 68 | F | C | 300 | Arm | LE: onset after | Symp | 18 | 0.4-3.1 | 10 | 15-75 | 27 | 0.3-4.4 | 18 | 13-89 |
| | | | | | | mastectomy | Asys | 38 | 0.3-1.6 | 14 | 12-140 | 67 | 0.2-2.2 | 41 | 14-177 |
| L06 | 18 | F | C | 400 | Leg | LE: onset at age 18 | Symp | 0 | N/A | 0 | N/A | 0 | N/A | 0 | N/A |
| | | | | | | with unknown | Asys | 3 | 0.6-1.5 | 0 | N/A | 7 | 0.3-3.1 | 3 | 18-28 |
| | | | | | | etiology (no trauma | | | | | | | | | |
| | | | | | | or surgery) | | | | | | | | | |
| L07 | 66 | F | C | 400 | Leg | LE: onset after vulvar | Symp | 11 | 0.2-0.8 | 6 | 44-113 | 7 | 0.5-1.0 | 4 | 55-207 |
| | | | | | | cancer surgery. | Asys | 15 | 0.6-1.3 | 7 | 26-201 | 6 | 0.5-0.8 | 2 | 27-36 |
| L08 | 23 | F | AA | 400 | Leg | LE: onset occurred | Symp | 0 | N/A | 0 | N/A | 9 | 0.9-3.7 | 6 | 10-67 |
| | | | | | | after bug bite | Asys | 6 | 0.2-1.1 | 2 | 42-43 | 8 | 0.3-0.5 | 4 | 20-113 |
| L09 | 48 | F | AA | 350 | Leg | LE: unknown etiology, | Symp | 2 | 0.5-0.5 | 1 | 60.4-60.4 | 11 | 0.4-2.4 | 7 | 37-68 |
| | | | | | | possibly due to | Asys | 3 | 0.6-1.6 | 0 | N/A | 10 | 0.4-2.5 | 5 | 10-38 |
| | | | | | | pressure from large, | | | | | | | | | |
| | | | | | | untreated fibroid | | | | | | | | | |
| | | | | | | cyst in uterine | | | | | | | | | |
| | | | | | | system. | | | | | | | | | |
| L10 | 40 | F | AA | 400 | Leg | LE: onset after spider | Symp | 15 | 0.1-1.4 | 6 | 6-228 | 13 | 0.1-1.1 | 7 | 15-120 |
| | | | | | | bite | Asys | 20 | 0.1-0.8 | 8 | 12-230 | 25 | 0.1-1.7 | 14 | 15-192 |
| | | | | | | on toe, also had fibroid | | | | | | | | | |
| | | | | | | cysts in groin | | | | | | | | | |
| N01 | 36 | F | C | 200 | Arm | Control normal | Left | 112 | 0.2-4.3 | 53 | 2-85 | 58 | 0.3-2.8 | 19 | 15-78 |
| | | | | | | | Right | 36 | 0.3-2.3 | 18 | 13-117 | 110 | 0.4-2.6 | 36 | 8-88 |
| N02 | 48 | F | C | 200 | Arm | Control normal | Left | 37 | 0.3-1.6 | 10 | 8-133 | 88 | 0.4-2.7 | 62 | 4-178 |
| | | | | | | | Right | 75 | 0.4-1.7 | 42 | 9-173 | 73 | 0.4-5.4 | 38 | 4-108 |
| N03 | 35 | F | AA | 200 | Arm | Control normal | Left | 12 | 0.2-0.7 | 3 | 41-55 | 21 | 0.4-1.2 | 15 | 12-135 |
| | | | | | | | Right | 15 | 0.2-0.8 | 6 | 31-57 | 90 | 0.3-0.9 | 69 | 7-178 |
| N04 | 41 | F | H | 300 | Arm | Control normal | Left | 61 | 0.2-2.4 | 33 | 13-78 | 55 | 0.2-2.1 | 21 | 18-69 |
| | | | | | | | Right | 48 | 0.1-1.5 | 28 | 16-72 | 130 | 0.2-1.8 | 71 | 3-86 |
| N05 | 35 | F | C | 300 | Arm | Control normal | Left | 21 | 0.3-1.1 | 15 | 27-209 | 16 | 0.2-1.0 | 4 | 37-142 |
| | | | | | | | Right | 131 | 0.2-1.0 | 107 | 16-189 | 46 | 0.4-1.0 | 36 | 15-160 |
| N06 | 37 | M | A | 300 | Arm | Control normal | Left | 75 | 0.4-1.0 | 51 | 7-205 | 53 | 0.2-1.3 | 32 | 14-183 |
| | | | | | | | Right | 175 | 0.1-1.2 | 115 | 9-251 | 62 | 0.2-1.3 | 37 | 8-258 |
| N07 | 35 | F | C | 400 | Leg | Control normal | Left | 5 | 0.8-1.2 | 0 | N/A | 13 | 0.5-1.5 | 10 | 33-178 |
| | | | | | | | Right | 19 | 0.3-2.1 | 7 | 16-85 | 17 | 1.0-2.1 | 3 | 27-67 |
| N08 | 40 | F | A | 400 | Leg | Control normal | Left | 35 | 0.2-2.3 | 15 | 7-125 | 71 | 0.1-2.3 | 51 | 6-140 |
| | | | | | | | Right | 45 | 0.3-1.9 | 29 | 14-110 | 90 | 0.2-8.3 | 68 | 2-122 |
| N09 | 25 | F | C | 400 | Leg | Control normal | Left | 42 | 0.2-2.5 | 21 | 3-194 | 12 | 0.5-1.9 | 4 | 15-42 |
| | | | | | | | Right | 3 | 0.3-1.1 | 0 | N/A | 5 | 0.7-2.4 | 3 | 54-90 |
| N10 | 34 | F | C | 400 | Leg | Control normal | Left | 20 | 0.4-1.4 | 12 | 12-125 | 23 | 0.7-1.6 | 12 | 11-119 |
| | | | | | | | Right | 17 | 0.3-1.8 | 5 | 33-110 | 19 | 0.6-1.3 | 8 | 17-105 |
| N11 | 22 | M | C | 400 | Leg | Control normal | Left | 30 | 0.1-1.7 | 10 | 28-107 | 55 | 0.3-2.6 | 34 | 10-172 |
| | | | | | | | Right | 12 | 0.4-1.3 | 3 | 50-107 | 27 | 0.3-1.8 | 12 | 17-139 |
| N12 | 59 | F | C | 400 | Leg | Control normal | Left | 36 | 0.4-2.7 | 12 | 28-83 | 94 | 0.5-12.0 | 66 | 5-214 |
| | | | | | | | Right | 33 | 0.1-11.7 | 9 | 5-153 | 43 | 0.1-2.5 | 10 | 14-254 |

*A = Asian, AA = African American, C = Caucasian, and H = Hispanic

TABLE 6
Pooled Data Statistics

| | Pre-MLD | | | | Post-MLD | | | | | | | |
|---|---|---|---|---|---|---|---|---|---|---|---|---|
| | | Mean and | Number | Mean and | | Mean and | Number | Mean and | | | | |
| | | standard | of | standard | | standard | of | standard | Post-vs. Pre-MLD | | | |
| Group | Number | deviation | periods | deviation | Number | deviation | periods | deviation | Change | p-value | Change | p-value |
| (number | of pulses | of velocity | between | of period | of pulses | of velocity | between | of period | of mean | of | of mean | of |
| of limb) | (velocity) | (cm/s) | pulses | (s) | (velocity) | (cm/s) | pulses | (s) | velocity | velocity | period | period |
| Symp | 55 | 1.04 ± 0.81 | 38 | 29.9 ± 18.0 | 85 | 1.19 ± 0.69 | 54 | 29.6 ± 24.8 | +13.6% | 0.054 | −1.1% | 0.376 |
| Arm (5) | | | | | | | | | | | | |
| Asys Arm | 129 | 0.68 ± 0.29 | 60 | 42.1 ± 31.4 | 293 | 0.87 ± 0.34 | 170 | 33.8 ± 24.7 | +29.1% | <0.001 | −19.8% | 0.092 |
| (5) | | | | | | | | | | | | |
| Control | 798 | 0.70 ± 0.32 | 481 | 56.6 ± 40.5 | 802 | 0.80 ± 0.40 | 440 | 41.6 ± 32.3 | +15.4% | <0.001 | −26.6% | <0.001 |
| Arm (12) | | | | | | | | | | | | |
| Symp Leg | 28 | 0.62 ± 0.32 | 13 | 73.6 ± 60.9 | 40 | 0.95 ± 0.66 | 24 | 55.4 ± 45.4 | +52.7% | 0.015 | −24.7% | 0.676 |
| (5) | | | | | | | | | | | | |
| Asys Leg | 47 | 0.71 ± 0.35 | 17 | 70.8 ± 65.5 | 56 | 0.76 ± 0.59 | 28 | 70.6 ± 53.5 | +7.4% | 0.954 | −0.2% | 0.971 |
| (5) | | | | | | | | | | | | |
| Control | 297 | 0.94 ± 0.80 | 123 | 53.9 ± 37.4 | 469 | 1.27 ± 1.11 | 281 | 46.3 ± 40.0 | +35.4% | <0.001 | −14.1% | 0.003 |
| Leg (12) | | | | | | | | | | | | |
| Symp (10) | 83 | 0.90 ± 0.71 | 51 | 41.0 ± 38.7 | 125 | 1.11 ± 0.68 | 78 | 37.5 ± 34.4 | +23.2% | 0.003 | −8.6% | 0.513 |
| Asys (10) | 176 | 0.68 ± 0.31 | 77 | 48.4 ± 42.9 | 349 | 0.85 ± 0.39 | 198 | 39.0 ± 32.9 | +24.9% | <0.001 | −19.5% | 0.069 |
| Control | 1095 | 0.76 ± 0.51 | 604 | 56.1 ± 39.9 | 1271 | 0.98 ± 0.78 | 721 | 43.4 ± 35.5 | +28.0% | <0.001 | −22.6% | <0.001 |
| (24) | | | | | | | | | | | | |

TABLE 7
Molecular Weight And Fluorescence Optical Properties of NIR Agents

| | | | Extinction | | | Intensity |
|---|---|---|---|---|---|---|
| | | | Coefficient × | Quantum | | relative to |
| Agent | MW | Excitation/Emission | 10⁴ | Yield | Lifetime | ICG |
| ICG | 775 | 785/799 | 8.60 +/− 0.35 | 0.016 | 0.59 +/− 0.049 | — |
| IRDye800 | 1166 | 774/789 | 57.11 +/− 0.09 | 0.053 +/− 0.004 | 0.93 +/− 0.075 | 21.8 |
| IRDye800-MSA | 39900 | 787/802 | | | | |
| IRDye800-ABD | 3441 | 780/803 | 54.836 +/− 0.25 | 0.0647 +/− 0.0007 | 0.92 +/− 0.057 | 25.9 |
| Dylight800 | 1050 | 770/794 | 19.256 +/− 0.19 | 0.0761 +/− 0.0005 | 0.88 +/− .0707 | 10.7 |

What is claimed is:

1. A method of non-invasively assessing lymph function in an individual, comprising: performing functional NIR fluorescence imaging of at least one lymphatic structure in said individual.
